SEQUENCE LISTING (9 sequences)

SEQ ID NO: 1 - DNA, 26 bp, synthetic construct
  source 1..26: mol_type = other DNA
ggaaaatgtaaagacaacgaatacag

SEQ ID NO: 2 - DNA, 24 bp, synthetic construct
  source 1..24: mol_type = other DNA
gctatcacataatctggaagcgta

SEQ ID NO: 3 - DNA, 17 bp, synthetic construct
  source 1..17: mol_type = other DNA
aagccgtaatctatgtt

SEQ ID NO: 4 - DNA, 23 bp, synthetic construct
  source 1..23: mol_type = other DNA
aatggcgttgacaattatgggtg

SEQ ID NO: 5 - DNA, 28 bp, synthetic construct
  source 1..28: mol_type = other DNA
attggtcattatttttgtcacaggaaca

SEQ ID NO: 6 - DNA, 14 bp, synthetic construct
  source 1..14: mol_type = other DNA
agagattagaaata

SEQ ID NO: 7 - DNA, 19 bp, synthetic construct
  source 1..19: mol_type = other DNA
agatttggacctgcgagcg

SEQ ID NO: 8 - DNA, 20 bp, synthetic construct
  source 1..20: mol_type = other DNA
gagcggctgtctccacaagt

SEQ ID NO: 9 - DNA, 23 bp, synthetic construct
  source 1..23: mol_type = other DNA
ttctgacctgaaggctctgcgcg

(19) United States
(12) Patent Application Publication
(10) Pub. No.: US 2024/0376557 A1
(43) Pub. Date: Nov. 14, 2024

(54) MOLECULAR TEST FOR MONKEYPOX VIRUS

(71) Applicants: Shuo Shen, Livermore, CA (US); Jonathan C Li, Danville, CA (US); Rui Ni, Richmond, CA (US); Michael Y Sha, Castro Valley, CA (US); Aiguo Zhang, San Ramon, CA (US)

(72) Inventors: Shuo Shen, Livermore, CA (US); Jonathan C Li, Danville, CA (US); Rui Ni, Richmond, CA (US); Michael Y Sha, Castro Valley, CA (US); Aiguo Zhang, San Ramon, CA (US)

(21) Appl. No.: 18/417,369

(22) Filed: Jan. 19, 2024

Related U.S. Application Data
(60) Provisional application No. 63/439,918, filed on Jan. 19, 2023.

(51) Int. Cl. C12Q 1/70

(52) U.S. Cl. CPC C12Q 1/701; C12Q 2600/156

(57) ABSTRACT

The present invention provides synthetic nucleic acid sequences comprising 10-30 nucleotides of the J2L and B6R gene regions and/or the 3′ non-coding region of the Monkeypox virus genome, and a synthetic nucleic acid sequence comprising 10-30 nucleotides of a nucleic acid sequence that is complementary to at least one of those regions. Also provided are compositions comprising the sequences, and uses of the sequences in diagnostic kits. The present invention further provides a primer and probe set for determining the presence or absence of Monkeypox virus in a biological sample, wherein the primer set comprises at least one of the synthetic nucleic acid sequences. Also provided are a composition comprising the primer and probe set, and use of the primer and probe set in a diagnostic kit. Finally, the present invention provides kits and methods for determining the presence or absence of Monkeypox virus in a biological sample.

This application claims the priority benefit under 35 U.S.C. section 119 of U.S. provisional Patent Application No. 63/439,918 entitled “Molecular Test For Monkeypox Virus” filed on Jan. 19, 2023; and which is in its entirety herein incorporated by reference.

### FIELD OF THE INVENTION

The present invention relates generally to the field of medicine and more specifically to infectious diseases. The invention also relates to the field of molecular biology, more particular to the detection of viral material in a biological sample.

The present invention relates to reagents and methods for detecting monkeypox (mpox) virus, in particular to reagents and methods for detecting monkeypox virus using amplification technology.

The present invention features a method for the detection of monkeypox virus in biological samples of living beings and to a kit for carrying out said method.

The present invention is also related to nucleic acid sequences that can be used in the field of virus diagnostics, more specifically the diagnosis of infections with monkeypox virus.

The present invention further relates to PCR primers and Taqman probes for detecting monkeypox virus, and a method and a kit for detecting monkeypox virus. The instant invention also relates to a quantitative real time RT-PCR method for detecting monkeypox virus and to oligonucleotides and kits for detecting monkeypox virus.

The present invention additionally relates to nucleic acid sequences that can be used in the field of virus diagnostics, more specifically the diagnosis of infections with monkeypox virus.

### BACKGROUND OF THE INVENTION

Monkeypox virus, first discovered in laboratory monkeys in 1958, can infect both animals and humans. Monkeypox virus belongs to the Orthopoxvirus genus in the family Poxviridae. The Orthopoxvirus genus also includes variola virus (which causes smallpox), vaccinia virus (used in the smallpox vaccine), and cowpox virus. Since the first human case of monkeypox infection was recorded in 1970, the majority of reported cases have been in Democratic Republic of the Congo and other central and western African countries. Recently, multiple cases have been reported in countries that do not normally report monkeypox infections, including Australia and countries in Europe and North America. In 2003, there was an outbreak in Wisconsin, USA, causing 82 deaths. people infected.

The exact mpox virus reservoir is unknown, although multiple species are carriers, including rodents and primates. Transmission to humans is typically related to direct contact with infected animals, body fluids, or respiratory droplets, with some evidence supporting aerosol and fomite transmission. The cessation of vaccinia vaccination, waning immunity following smallpox eradication, and increased human migration have increased the risk of mpox and catalyzed the recent global outbreaks.

Monkeypox virus is a double-stranded DNA virus. It belongs to the Poxviridae family like smallpox virus. Symptoms of infection are similar to smallpox, such as fever, headache, swollen lymph nodes, cough and extreme pain all over the body. It is commonly known as “monkeypox” and is not imported into my country. One of the dangerous viruses, it can be transmitted through direct contact with patients or infected animals, or through the body fluids of patients. Highly virulent strains of the virus may be fatal.

MPXV is known to have a wide-reaching host tropism and can infect many different species. This generality also translates into its cell and tissue tropisms; the virus has been found to infect tissues ranging from the heart and brain to the ovaries and lymphoid tissue.

Once inside the body, MPXV infects cells through a series of interactions between viral and cellular proteins, for instance the viral D8L protein, which binds to the cell surface receptor chondroitin sulfate. Once bound, the virus enters the cell by fusing with the cell membrane or by endocytosis. In this manner, the virus can enter cells, replicate and then infiltrate the bloodstream, after which it can spread through the bloodstream to any of the many tissue types that it is capable of infecting.

The monkeypox genome is a large single linear molecule of dsDNA, about 197 kilobase-pairs (kbp) in length. The genome consists of about 190 non-overlapping open reading frames (>180 bp long) containing 60 or more amino acid residues. There are two clades of MPXV: the West African clade (clade II) and the Congo Bastin clade (clade I). Applicants Quanti Virus MPXV Test Kit detects DNA from both clade I and clade II of MPXV in lesion swab specimens (i.e., swabs of acute pustular or vesicular rash). It uses a fluorescent probe with specific primer sets to detect the J2L and B6R genes within the genome of MPXV. Primers and probe for an internal control, RNase P are also integrated in the assay to validate the assay quality.

Orthopoxvirus infections are difficult to characterize by serology due to cross-reactivity between other genus members, making molecular testing by polymerase chain reaction (PCR) the preferred diagnostic method for mpox, given its accuracy and sensitivity. Mpox also differs from other viral pathogens in that standard specimens needed for accurate diagnosis are skin lesions, dry crusts, or biopsy, instead of the more typical and accessible blood, serum, and sputum samples, meaning that PCR-based diagnostics are only effective when samples are taken during the active rash phase of the illness. The recent influx of mpox cases and the difficulty of diagnosing atypical infections outside of endemic regions highlights the need for rapid identification to assist with diagnosis and case management to decrease further community spread. Molecular dating, phylogenetic, and coding region analysis can assist with understanding potential transmission chains and epidemiological factors. Furthermore, sequencing data assists medical countermeasure development, suitability, and effectiveness. However, the current laboratory infrastructures where mpox is endemic need improvement for effective surveillance, treatment, and prevention of the disease, including portable molecular testing capabilities.

Monkeypox symptoms often resolve on their own, and care focuses on alleviating symptoms. No therapeutics have been developed for treating monkeypox specifically; however, due to the similarities between monkeypox and smallpox, treatments that were originally developed for smallpox could potentially be used to treat monkeypox. For example, tecovirimat is an antiviral with activity against smallpox, monkeypox and other orthopoxviruses. The drug is approved for treatment of smallpox in the USA and is approved for the treatment of both smallpox and monkeypox in the European Union, although it is not yet widely available.

### SUMMARY OF THE INVENTION

The invention provides a PCR primer set useful for detecting Monkeypox virus selected from the group consisting of the following primer sets: (a) a primer set comprising a primer consisting of MPXVF SEQ ID NO: 1 GGAAAATGTAAAGACAACGAATACAG and a primer MPXVR SEQ ID NO: 2 GCTATCACATAATCTGGAAGCGTA; (b) a primer set comprising a primer consisting of B6RF SEQ ID NO: 4 AATGGCGTTGACAATTATGGGTG and a primer consisting of B6RR SEQ ID NO: 5 ATTGGTCATTATTTTTGTCACAGGAACA; and (c) a primer set comprising a primer consisting of RNasePF SEQ ID NO: 7 AGATTTGGACCTGCGAGCG and a primer consisting of RNasePR SEQ ID NO: 8 GAGCGGCTGTCTCCACAAGT; wherein the primer set specifically amplifies a target region of Monkeypox virus in a polymerase chain reaction (PCR).

The invention also provides Oligonucleotides, for use as a probe to detect the amplified nucleic acid sequence resulting in the amplification of a target sequence located within the genome of Monkeypox virus, said probe being selected from the group consisting of MPXVPr (Probe) SEQ ID NO: 3 AAGCCGTAATC TATGTT; B6RPr (Probe); SEQ ID NO: 6 AGAGATTAGAAATA and RNasePPr (Probe) SEQ ID NO: 9 TTCTGACCTGAAGGCTCTG CGCG.

The invention further provides a method for detecting Monkeypox virus by contacting a biological sample with a set of primers and a probe, incubating under conditions allowing amplification of nucleic acid using said primers, and determining binding of said probe to amplified nucleic acid, wherein detecting binding of said probe to amplified nucleic acid indicates the presence of Monkeypox associated virus, wherein the the primers are selected from the group consisting of the following primer sets: (a) a primer set comprising a primer consisting of MPXVF SEQ ID NO: 1 GGAAAATGTAAAGACAACGAATACAG and a primer MPXVR SEQ ID NO: 2 GCTATCACATAATCTGGAAGCGTA; (b) a primer set comprising a primer consisting of B6RF SEQ ID NO: 4 AATGGCGTTGACAATTATGGGTG and a primer consisting of B6RR SEQ ID NO: 5 ATTGGTCATTATTTTTGTCACAGGAACA; and (c) a primer set comprising a primer consisting of RNasePF SEQ ID NO: 7 AGATTTGGACCTGCG AGCG and a primer consisting of RNasePR SEQ ID NO: 8 GAGCGGCTGTCTCCACAAGT; and wherein the probe is selected from the group consisting of MPXVPr (Probe) SEQ ID NO: 3 AAGCCGTAATC TATGTT; B6RPr (Probe); SEQ ID NO: 6 AGAGATTAGAAATA and RNasePPr (Probe) SEQ ID NO: 9 TTCTGACCTGAAGGCTCTGCGCG; and wherein the probe is labeled with two dyes, one dye of which is a fluorescent reporter dye, and one dye of which is a quencher dye, and wherein at least one dye is a fluorescent dye; and the Monkeypox virus is detected by detection of real time fluorescence, if amplification of virus specific sequence occurs.

Additionally, the invention also relates to a kit for detecting Monkeypox virus in a biological sample comprising a PCR primer set selected from the group consisting of the following primer sets: (a) a primer set comprising a primer consisting of MPXVF SEQ ID NO: 1 GGAAAATGTAAAGACAACGAATACAG and a primer MPXVR SEQ ID NO: 2 GCT ATCACATAATCTGGAAGCGTA; (b) a primer set comprising a primer consisting of B6RF SEQ ID NO: 4 AATGGCGTTGACAATTATGGGTG and a primer consisting of B6RR SEQ ID NO: 5 ATTGGTCATTATTTTTGTCACAGGAACA; and (c) a primer set comprising a primer consisting of RNasePF SEQ ID NO: 7 AGATTTGGACCTGCGAGCG and a primer consisting of RNasePR SEQ ID NO: 8 GAGCGGCTGTCTCCACAAGT; wherein the primer set specifically amplifies a target region of Monkeypox virus in a polymerase chain reaction (PCR).

The kits of the invention further include a probe selected from the group consisting of MPXVPr (Probe) SEQ ID NO: 3 AAGCCGTAATC TATGTT; B6RPr (Probe); SEQ ID NO: 6 AGAGATTAGAAATA and RNasePPr (Probe) SEQ ID NO: 9 TTCTGACCTGAAGGCTC TGCGCG; as well as a reporter dye selected from the group consisting of FAM, 6-FAM, 5-FAM and ALEXA-288; and a quencher dye selected from the group consisting of TAMRA, DABCYL or QSY.

According to another aspect of the present invention, there is provided a method for detecting monkeypox virus, which includes amplifying a nucleic acid sample obtained from an individual by PCR using the primers and probes of the invention.

According to yet another aspect of the present invention, there is provided a monkeypox detection kit including the primers and probes of the invention.

### BRIEF DESCRIPTION OF THE FIGURES

FIG. 1 illustrates MPXV viral DNA isolation and detection workflow of 384 patient samples in the average run duration of 2.5-3 hours.

FIG. 2 shows the steps for performing the assay of the invention.

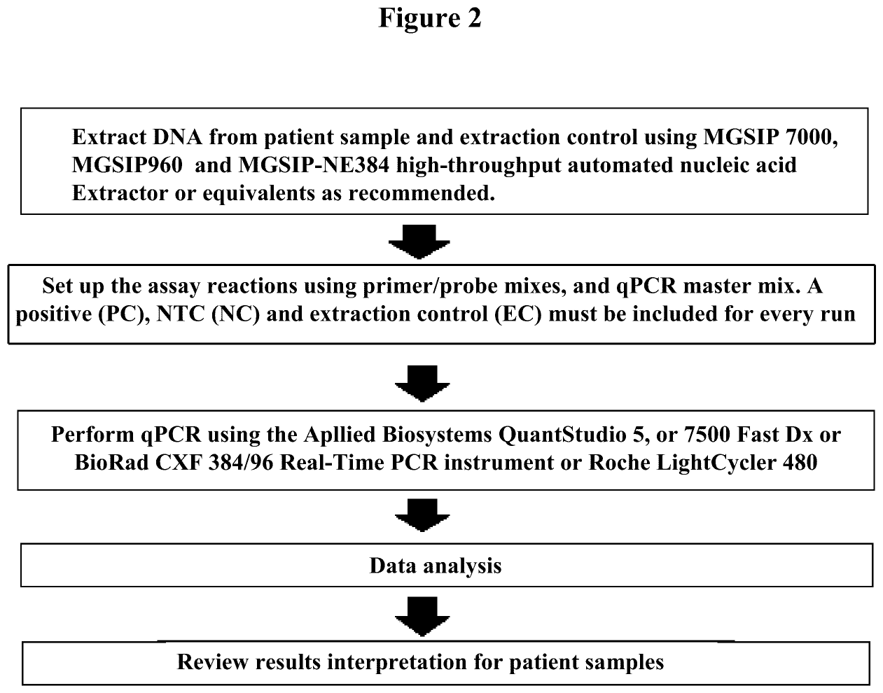

FIG. 3. fetaures the amplification Curve of 10-fold serial dilution of templates showing the threshold setting.

REFERENCE TO SEQUENCE LISTING

The present application is being filed along with a Sequence Listing in electronic format. The Sequence Listing is provided as a file entitled “Dia045Sequencelisting.xml”, created Jan. 19, 2024, which is 8,977 bytes in size. The information in the electronic format of the Sequence Listing is incorporated herein by reference in its entirety.

### DETAILED DESCRIPTION OF THE INVENTION

Whereas conventional virus diagnosis has been based predominantly on the detection of viral antigens or specific antibodies thereto, in recent years attention has shifted towards methods for the direct and rapid detection of the genome of viruses or nucleic acid sequences derived thereof, both RNA and DNA. In this respect, the very short time-to-result is a crucial factor to opt for nucleic acid detection. These methods are usually based on nucleic acid hybridization. Nucleic acid hybridization is based on the ability of two strands of nucleic acid containing complementary sequences to anneal to each other under the appropriate conditions, thus forming a double stranded structure. When the complementary strand is labeled, the label can be detected and is indicative for the presence of the target sequence. Especially in combination with methods for the amplification of nucleic acid sequences these methods have become an important tool in viral diagnosis.

Nucleic acid amplification techniques are especially useful as an additional technique in cases where serological methods give doubtful results or in cases where there may be a considerable time period between infection and the development of antibodies to the virus.

The choice of the oligonucleotides to be used as primers and probes in the amplification and detection of nucleic acid sequences is critical for the sensitivity and specificity of the assay. The sequence to be amplified is usually only present in a sample (for example a blood sample obtained from a patient suspected of having a viral infection) in minute amounts. The primers should be sufficiently complementary to the target sequence to allow efficient amplification of the viral nucleic acid present in the sample. If the primers do not anneal properly (due to mispairing of the bases on the nucleotides in both strands) to the target sequence, amplification is seriously hampered. This will affect the sensitivity of the assay and may result in false negative test results. Due to the heterogeneity of viral genomes false negative test results may be obtained if the primers and probes are capable of recognizing sequences present in only part of the variants of the virus.

The invention provides a PCR primer set useful for detecting Monkeypox virus selected from the group consisting of the following primer sets: (a) a primer set comprising a primer consisting of MPXVF SEQ ID NO: 1 GGAAAATGTAAAGACAACGAATACAG and a primer MPXVR SEQ ID NO: 2 GCTATCACATAATCTGGAAGCGTA; (b) a primer set comprising a primer consisting of B6RF SEQ ID NO: 4 AATGGCGTTGACAATTATGGGTG and a primer consisting of B6RR SEQ ID NO: 5 ATTGGTCATTATTTTTGTCACAGGAACA; and (c) a primer set comprising a primer consisting of RNasePF SEQ ID NO: 7 AGATTTGGACCTGCGAGCG and a primer consisting of RNasePR SEQ ID NO: 8 GAGCGGCTGTCTCCACAAGT; wherein the primer set specifically amplifies a target region of Monkeypox virus in a polymerase chain reaction (PCR).

The invention also provides Oligonucleotides, for use as a probe to detect the amplified nucleic acid sequence resulting in the amplification of a target sequence located within the genome of Monkeypox virus, said probe being selected from the group consisting of MPXVPr (Probe) SEQ ID NO: 3 AAGCCGTAATC TATGTT; B6RPr (Probe); SEQ ID NO: 6 AGAGATTAGAAATA and RNasePPr (Probe) SEQ ID NO: 9 TTCTGACCTGAAGGCTCTG CGCG.

The invention further provides a method for detecting Monkeypox virus by contacting a biological sample with a set of primers and a probe, incubating under conditions allowing amplification of nucleic acid using said primers, and determining binding of said probe to amplified nucleic acid, wherein detecting binding of said probe to amplified nucleic acid indicates the presence of Monkeypox associated virus, wherein the the primers are selected from the group consisting of the following primer sets: (a) a primer set comprising a primer consisting of MPXVF SEQ ID NO: 1 GGAAAATGTAAAGACAACGAATACAG and a primer MPXVR SEQ ID NO: 2 GCTATCACATAATCTGGAAGCGTA; (b) a primer set comprising a primer consisting of B6RF SEQ ID NO: 4 AATGGCGTTGACAATTATGGGTG and a primer consisting of B6RR SEQ ID NO: 5 ATTGGTCATTATTTTTGTCACAGGAACA; and (c) a primer set comprising a primer consisting of RNasePF SEQ ID NO: 7 AGATTTGGACCTGCG AGCG and a primer consisting of RNasePR SEQ ID NO: 8 GAGCGGCTGTCTCCACAAGT; and wherein the probe is selected from the group consisting of MPXVPr (Probe) SEQ ID NO: 3 AAGCCGTAATC TATGTT; B6RPr (Probe); SEQ ID NO: 6 AGAGATTAGAAATA and RNasePPr (Probe) SEQ ID NO: 9 TTCTGACCTGAAGGCTCTGCGCG; and wherein the probe is labeled with two dyes, one dye of which is a fluorescent reporter dye, and one dye of which is a quencher dye, and wherein at least one dye is a fluorescent dye; and the Monkeypox virus is detected by detection of real time fluorescence, if amplification of virus specific sequence occurs.

Additionally, the invention also relates to a kit for detecting Monkeypox virus in a biological sample comprising a PCR primer set selected from the group consisting of the following primer sets: (a) a primer set comprising a primer consisting of MPXVF SEQ ID NO: 1 GGAAAATGTAAAGACAACGAATACAG and a primer MPXVR SEQ ID NO: 2 GCT ATCACATAATCTGGAAGCGTA; (b) a primer set comprising a primer consisting of B6RF SEQ ID NO: 4 AATGGCGTTGACAATTATGGGTG and a primer consisting of B6RR SEQ ID NO: 5 ATTGGTCATTATTTTTGTCACAGGAACA; and (c) a primer set comprising a primer consisting of RNasePF SEQ ID NO: 7 AGATTTGGACCTGCGAGCG and a primer consisting of RNasePR SEQ ID NO: 8 GAGCGGCTGTCTCCACAAGT; wherein the primer set specifically amplifies a target region of Monkeypox virus in a polymerase chain reaction (PCR).

According to another aspect of the present invention, there is provided a method for detecting monkeypox virus, which includes amplifying a nucleic acid sample obtained from an individual by PCR using the primers and probes of the invention.

According to yet another aspect of the present invention, there is provided a monkeypox detection kit including the primers and probes of the invention.

The kits of the invention further include a probe selected from the group consisting of MPXVPr (Probe) SEQ ID NO: 3 AAGCCGTAATC TATGTT; B6RPr (Probe); SEQ ID NO: 6 AGAGATTAGAAATA and RNasePPr (Probe) SEQ ID NO: 9 TTCTGACCTGAAGGCTC TGCGCG; as well as a reporter dye selected from the group consisting of FAM, 6-FAM, 5-FAM and ALEXA-288; and a quencher dye selected from the group consisting of TAMRA, DABCYL or QSY.

As used herein, the term “PCR” is well known in the pertinent art. Generally, PCR includes the steps of: (a) obtaining a crude extract containing target cDNA or DNA molecules from a sample; (b) adding an aqueous solution including an enzyme, a buffer, dNTPs, and oligonucleotide primers to the crude extract; (c) amplifying the target DNA molecules by two-or three-step thermal cycling (e.g., 90-96° C., 72° C., and 37-55° C.) of the resultant mixture; and (d) detecting amplified DNAs. In the present invention, the PCR may be performed in a polypropylene tube, a 96-well plate, or a silicon-based micro PCR chip.

When the PCR is performed on a silicon-based micro PCR chip, a two-step thermal cycling as well as a three-step thermal cycling can be used. A time required for the PCR on the silicon-based micro PCR chip can be as short as 30 minutes or less. For example, the silicon-based micro PCR chip includes a silicon wafer, a surface of which is formed with a PCR chamber made by silicon lithography and the other surface is formed with a heater for heating the PCR chamber; and a glass wafer having an inlet and an outlet.

In the present invention, the PCR may be performed using 0.2-1. mM of each primer and 0.01 pg to 1 mg of a template DNA.

In the present invention, the PCR may be performed in three-step thermal cycling conditions of denaturation at 86-97° C. for 1-30 seconds, annealing at 50-70° C. for 1-30 seconds, and extension at 60-72° C. for 1-30 seconds, or in two-step thermal cycling conditions of denaturation at 86-97° C. for 1-30 seconds and annealing and extension at 50-70° C. for 5-30 seconds.

In a preferred embodiment, Applicants use real-time polymerase chain reaction (real-time PCR), also known as quantitative polymerase chain reaction (qPCR), is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively (quantitative real-time PCR) and semi-quantitatively (i.e., above/below a certain amount of DNA molecules) (semi-quantitative real-time PCR).

Two common methods for the detection of PCR products in real-time PCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence.

As is commonly known, real-time PCR is carried out in a thermal cycler with the capacity to illuminate each sample with a beam of light of at least one specified wavelength and detect the fluorescence emitted by the excited fluorophore. The thermal cycler is also able to rapidly heat and chill samples, thereby taking advantage of the physicochemical properties of the nucleic acids and DNA polymerase.

The PCR process generally consists of a series of temperature changes that are repeated 25-50 times. These cycles normally consist of three stages: the first, at around 95° C., allows the separation of the nucleic acid's double chain; the second, at a temperature of around 50-60° C., allows the binding of the primers with the DNA template; the third, at between 68-72° C., facilitates the polymerization carried out by the DNA polymerase. Due to the small size of the fragments the last step is usually omitted in this type of PCR as the enzyme is able to increase their number during the change between the alignment stage and the denaturing stage. In addition, in four step PCR the fluorescence is measured during short temperature phase lasting only a few seconds in each cycle, with a temperature of, for example, 80° C., in order to reduce the signal caused by the presence of primer dimers when a non-specific dye is used. The temperatures and the timings used for each cycle depend on a wide variety of parameters, such as: the enzyme used to synthesize the DNA, the concentration of divalent ions and deoxyribonucleotides (dNTPs) in the reaction and the bonding temperature of the primers.

In the present invention, lesion swab specimens are collected in Viral Transport Media (VTM) or equivalent. A total of 200 μL of specimen is used for DNA isolation by a MGISP-NE384 High-throughput Automated Sample Preparation System. Detection of PCR amplicons is accomplished using TaqMan chemistry on the ABI QuantStudio 5, ABI 7500, Bio-Rad CFX 384 or Roche LightCycler 480 II. The assay detects the two gene targets within the MPXV multiplexed in one tube, along with human RNase P. The RNase P target is an internal control which can be evaluated for successful DNA extraction and PCR reaction.

### Material and Methods

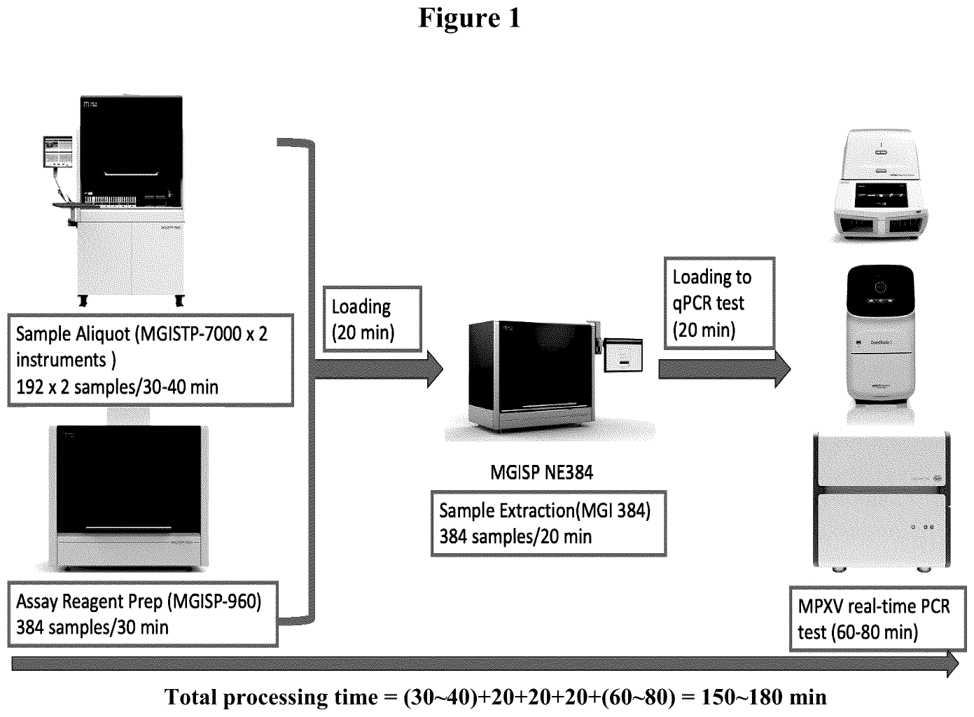

The Quanti Virus MPXV Test of the invention provides high-throughput technologies by using an automated sample preparation system and an automated nucleic acid extractor, along with 384-well PCR Thermal Cyclers. A total of 384 samples can be tested over 2.5-3 hours (FIG. 1). Therefore, for an 8-hour shift, a single operator should run 2-3 plates of 384 wells (768-1,152 patient samples) with one set of machines (two MGISTP 7000, one MGISP960, one MGISP-NE384 and one real-time PCR machine, Table 1). This high-throughput assay has already been validated for the Quanti Virus MPXV Test by the applicant CLIA lab. Depending on the volumes, labs can establish more sets of machines to increase the test throughput significantly. For example, the applicant CLIA lab owns 6 sets of these instruments and can potentially run up to 20,000 samples per day (three shifts) using Quanti Virus MPXV Test Kit of the invention.

MGISTP-7000 and MGISP-960 are an automated sample transfer processing system and an automated sample preparation system, respectively. MGISP-NE384 is able to extract and purify nucleic acid from 384 samples.

TABLE 1
Turnaround Time from Sample to Answer

| Procedural Steps | Time Required | |
|---|---|---|
| Aliquot of samples and reagents by 2x | 30-40 | min |
| MGISTP-7000 & MGISP-960 | | |
| Loading onto MGISP-NE 384 | 20 | min |
| MGISP-NE384 Automatic Viral DNA | 20 | min |
| Extraction of 384 Samples | | |
| Loading onto 384-well real-time thermal cyclers (i.e., | 20 | min |
| Bio-Rad CFX384, ABI QS5, or LightCycler 480 II) | | |
| PCR run | 60-80 | min |

| | |
|---|---|
| Total times for 384 patient samples | 150-180 min |
| | (2.5-3 hours) |

### Proposed Intended Use:

The Quanti Virus MPXV Test of the invention is a real-time PCR test intended for the qualitative detection of DNA from non-variola Orthopoxvirus/monkeypox virus in human skin lesion material specimens such as lesion exudate, lesion roofs or lesion crusts, etc. This test's intended use is for individuals suspected of Monkeypox by their healthcare provider. Testing is limited to laboratories certified under the Clinical Laboratory Improvement Amendments of 1988 (CLIA), 42 U.S.C. 63a, that meet the regulatory requirements to perform high complexity testing.

Results are for the identification of non-variola Orthopoxvirus or monkeypox virus DNA. The non-variola Orthopoxvirus or monkeypox virus DNA is generally detectable in samples such as lesion exudate, lesion roofs or lesion crusts, etc. during the acute phase of infection. Positive results are indicative of the presence of non-variola Orthopoxvirus or monkeypox virus DNA; clinical correlation with patient history and other diagnostic information is necessary to determine patient infection status. Positive results do not rule out bacterial infection or co-infection with other viruses. The agent detected may not be the definite cause of disease. Negative results obtained with this device do not preclude non-variola Orthopoxvirus or monkeypox virus infection, and should not be used as the sole basis for treatment or other patient management decisions. Negative results must be combined with clinical observations, patient history, and epidemiological information.

Laboratories within the United States and its territories are required to report test results to the appropriate public health authorities. The Quanti Virus MPXV Test of the invention is intended for use by qualified, and trained clinical laboratory personnel specifically instructed and trained in the techniques of PCR and in vitro diagnostic procedures.

Quanti Virus™ MPXV Test is only for use under the Food and Drug Administration's Emergency Use Authorization.

Instruments Required: ABI QS5, ABI 7500, BioRad CFX 384, Roche LC480II.

Primers/Probes: Applicant designed primers and probes targeted to MPXV J2L and BR6 gene and use human RNase P gene as internal control. Detailed sequences can be seen in Table 1 below.

TABLE 1
Primer and Probe Design for QuantiVirus MPXV Test

| Primer/Probe | | | MPXV |
|---|---|---|---|
| Name | Sequence | label Dye | Gene |
| MPXV F | SEQ ID NO: 1 GGAAAATGTAAAGACAACGAATACAG | | J2L |
| MPXV R | SEQ ID NO: 2 GCTATCACATAATCTGGAAGCGTA | | |
| MPXV Pr | SEQ ID NO: 3 AAGCCGTAATCTATGTT | FAM-MGB | |
| B6R F | SEQ ID NO: 4 AATGGCGTTGACAATTATGGGTG | | B6R |
| B6R R | SEQ ID NO: 5 ATTGGTCATTATTTTTGTCACAGGAACA | | |
| B6R Pr | SEQ ID NO: 6 AGAGATTAGAAATA | HEX-MGB | |
| RNaseP F | SEQ ID NO: 7 AGATTTGGACCTGCGAGCG | | human |
| RNaseP R | SEQ ID NO: 8 GAGCGGCTGTCTCCACAAGT | | Rnase |
| RNaseP Pr | SEQ ID NO: 9 TTCTGACCTGAAGGCTCTGCGCG | Cy5 | P |

A list of useful fluorescent dyes and quenchers for using with the probes are listed in Table 1a below.

TABLE 1a
Dyes and Quenchers usable with the invention

| | Max. | Max. | Compatible |
|---|---|---|---|
| Dye | EX (nm) | EM (nm) | Quencher |
| 6-FAM ™ | 494 | 515 | BHQ-1, DABCYL |
| Fluorescein | 495 | 520 | BHQ-1, DABCYL |
| JOE ™ | 520 | 548 | BHQ-1, DABCYL |
| TET | 521 | 536 | BHQ-1, DABCYL |
| Cal Fluor ® Gold 5401 | 522 | 541 | BHQ-1 |
| HEX | 535 | 555 | BHQ-1, DABCYL |
| Cal Fluor Orange 5602 | 540 | 561 | BHQ-1 |
| TAMRA ™ | 555 | 576 | BHQ-2 |
| Cyanine 3 | 550 | 570 | BHQ-2, DABCYL |
| Quasar ® 5703 | 548 | 566 | BHQ-2 |
| ROX ™ | 573 | 602 | BHQ-2, DABCYL |
| Texas Red ® | 583 | 603 | BHQ-2, DABCYL |
| Cyanine 5 | 651 | 674 | BHQ-3, DABCYL |
| Quasar 6705 | 647 | 667 | BHQ-3 |
| Cyanine 5.5 | 675 | 694 | BHQ-3, DABCYL |

### Test Steps:

The brief procedure for performing the assay includes the following steps:
1. Extract DNA from patient sample and extraction control using MGSIP 7000, MGSIP960 and MGSIP-NE384 high-throughput automated nucleic acid Extractor or equivalents as recommended.
2. Set up the assay reactions using primer/probe mixes, and qPCR master mix. A positive (PC), NTC (NC) and extraction control (EC) must be included for every run.
3. Perform qPCR using the Applied Biosystems QuantStudio 5, or 7500 Fast Dx or BioRad CXF 384/96 Real-Time PCR instrument or Roche LightCycler 480.
4. Data analysis
5. Review results interpretation for patient samples

The workflow begins with DNA extraction from lesion swab specimens. DNA is isolated and purified from the specimens using the appropriately chosen viral DNA extraction method. The purified DNA is amplified using Quanti Virus MPXV Test Kit of the invention on either ABI QuantStudio 5, ABI 7500 Fast Dx, Bio-Rad CXF 384/96, or Roche LightCylcer 480 II Real-Time PCR instrument. In the process, the probes anneal to the specific target sequences located between one pair of unique forward and reverse primers for the J2L and B6R genes in the MPXV genome. The RPP30's primers and probe target the human RNase P gene to monitor successful DNA extraction. During the extension phase of the PCR cycle, the 5′ exonuclease activity of Taq polymerase degrades the probe, causing the reporter dye to separate from the quencher dye, generating a fluorescent signal. With each cycle, additional reporter dye molecules are cleaved from their respective probes, increasing the fluorescence intensity. Fluorescence intensity is monitored at each PCR cycle by the PCR instrument.

### Controls Required:

Included with the Test Kit:

| | | | | Where it | Frequency |
|---|---|---|---|---|---|
| Control | | Requirement | How it works | is used | of use |
| Positive | A positive control is a mix of | This control | It has J2L and | Acceptable | Each test |
| | synthetic DNA templates for | must be used in | BR6 target | Cq must | must be |
| | the target sequences for J2L | order to monitor | sequence in the | be <30 | included |
| | and B6R genes of the MPXV | the whole qPCR | tube, each | | |
| | genome. Positive controls | test works well. | qPCR test will | | |
| | must show the appropriate | | show positive | | |
| | values in FAM and HEX | | amplification | | |
| | channel for the run to be | | curve. | | |
| | valid. Positive control | | | | |
| | monitors the function of each | | | | |
| | assay component | | | | |
| Negative | No Template Control (NTC) | This control | It does not | Acceptable | Each test |
| | Nuclease-free water is used | must be used in | have J2L and | Cq must | must be |
| | in place of template. No | order to monitor | BR6 target | be >45 | included |
| | amplification should be | the whole qPCR | sequence in the | | |
| | observed in all channels, | test works well | tube, each | | |
| | assuring the absence of | without any | qPCR test will | | |
| | contamination during assay | contamination | not show | | |
| | set-up | occurs | amplification | | |
| | | | curve. | | |
| Extraction | Extraction Control is a human | In order make | It has RPP30 | Acceptable | Each test |
| | RNase P (RPP30) gene DNA. | included for | gene target in | Cq must | must be |
| | The extraction control RP | sure that each | the tube and | be <38 | included |
| | DNA undergoes the full | extraction step | each qPCR | | |
| | extraction procedure. As the | work well, this | will show | | |
| | Extraction Control, there | extraction | amplification | | |
| | should be amplification for | control must be | curve. | | |
| | RP gene, but no amplification | each viral DNA | | | |
| | for the viral gene (J2L or | extraction | | | |
| | B6R). This control should be | procedures. | | | |
| | run with every batch of | | | | |
| | extraction | | | | |
| Internal | We use human RRP30 gene | In order to | Each sample | Acceptable | Each |
| | as internal control | confirm that | has RPP30 | Cq must | sample |
| | | each sample has | gene target and | be <38 | must be |
| | | enough DNA | each qPCR | | tested in |
| | | for testing. | will show | | order to be |
| | | | amplification | | valid |
| | | | curve. | | |

### Testing Capabilities

There are a total of four steps from sample extraction to result analysis: 1) aliquot of sample and reagents by MGISTP 7000 and MGISP960; 2). DNA extraction by MGISP-NE384; 3) qPCR running; and 4). data analysis. Generally speaking, MPXV viral DNA isolation and detection for 384 patient samples/2.6 hrs (Table 2).

TABLE 2
Turnaround Time from Sample to Answer

| Procedural Steps | Time Required | |
|---|---|---|
| Aliquot of samples and reagents by 2x MGISTP7000 & | 30 | minutes |
| MGISP 960 | | |
| Loading onto MGISP-NE 384 | 20 | minutes |
| MGISP-NE384 Automatic Viral DNA Extraction of 384 | 20 | minutes |
| Samples | | |
| Loading onto BioRad CFX384 or ABI QS5 | 20 | minutes |
| MPXV qPCR, 384 wells | 60 | minutes |
| Result analysis for 384 patient samples | 10 | minutes |
| Total times for 384 patient samples | 160 | minutes |

Number of patient tests that can be performed per day (8-hr shift): 1,152 patient samples can be tested with one set of instruments and one trained lab user during an 8-hr shift. DiaCarta CLIA lab owns 6 sets of these instruments and can potentially run up to 20,000 samples per day (three shifts) using Quanti Virus MPXV Test Kit of the invention.

Device Components and Components Included with the Test:

The Quanti Virus MPXV Test kit of the invention includes the following components:
1. qPCR Master mix
2. One set of Primers/Probe specific to the J2L& B6R Monkeypox genomic region and primers/probe for human RNase P gene.
3. A Positive control (PC), Extraction control (EC) and a No Template control (NTC).

The Quanti Virus MPXV Test kit of the invention can be made in 3 pack sizes—24-reactions kit, 48-reaction kit and 480-reaction kit. Individual components and their descriptions are listed in Table 3 below.

TABLE 3a
Kit components Pack-Size: 24 Reactions

| | | Pack | Label | |
|---|---|---|---|---|
| | | Size: 24 | Volume | |
| Name of | | reactions | for each | Storage |
| Component | Description | kit | vial | Temp |
| Primer/Probe | Primer/probe Mix (J2L, B6R & | 1 vial | 48 μL | −25° C. |
| Mix | Human Rnase P gene primers and | | | to −15° C. |
| | probes) | | | |
| Master Mix | Meridian Inhibitor-Tolerant | 1 vial | 48 μL | −25° C. |
| | Master mix | | | to −15° C. |
| Positive | Synthetic DNA templates (Positive | 1 vial | 10 μL | −25° C. |
| Controls | control PC) for J2L & B6R | | | to −15° C. |
| Extraction | Human Specimen Extraction | 1 vial | 40 μL | −25° C. |
| Control (EC) | Control | | | to −15° C. |
| No Template | Nuclease-Free Water | 1 vial | 50 μL | −25° C. |
| Control | | | | to −15° C. |

See Table 4 for the qualitative detection of monkeypox viral genes J2L and B6R. Instructions for use for additional information:
1). QuantStudio5 Real-Time PCR System, 384-well Catalog number: A28140 See www.thermofisher.com/order/catalog/product/A28140?SID-srch-srp-A28140.
2) BioRad CXF 384 Real-Time PCR Instrument. Cat #1855484 See www.bio-rad.com/en-us/product/cfx384-touch-real-time-pcr-detection-systemID=LJB22YE8Z
3). Roche LightCycler 480 II, Cat #5125-00-1113 See https://sequencing.roche.com/us/en/products/group/lightcycler-480-ii.html.
4). Applied Biosystems 7500 Fast Dx Real-Time PCR Instrument, Catalog number: 4406985 See www.thermofisher.com/order/catalog/product/4406985?SID=srch-hj-4406985
5). BioRad CXF 96 Real-Time PCR Instrument, cat #1845097. See www.bio-rad.com/en-us/product/cfx96-touch-real-time-pcr-detection-system?ID=LJB1YU15.
   This instrument operation manual addendum applies to the instruments listed in Table 3 that are for use with the Quanti Virus MPXV Test.

TABLE 4
Instruments Authorized for Emergency Use
Only with the QuantiVirus MPXV Test.

| Catalog Number | Product Name |
|---|---|
| Thermos Fisher Scientific Cat# | Applied Biosystems ™ QuantStudio 5 |
| A28140 | Real-Time PCR Instrument |
| BioRad Cat# 1855484 | BioRad CXF 384 Real-Time PCR |
| | Instrument |
| Roche Cat# 5125-00-1113 | Roche LightCycler 480 II |
| Thermos Fisher Scientific Cat# | ABI 7500 Fast DX |
| 4406985 | |
| BioRad Cat#1845097 | BioRad CXF 96 Real-Time PCR |
| | Instrument |

### Viral DNA Isolation

### 1) Automated High-Throughput Extraction

The Quanti Virus MPXV Test Kit of the invention uses MGISP-NE384 automated high-throughput nucleic acid extractor and MGIEasy® Nucleic Acid Extraction Kit (cat #1000020261)
   Use MGISTP-7000 to aliquot MPXV samples into 96-well deep well plates.
   While aliquoting samples, prepare the extraction reagents (MLB MIX, MW1, MW2, and Elution water) with MGISP-960.
   Add MLB into 96-well plates with samples.
   Load the samples and extraction reagents into MGISP-NE384 as follows:

| Reagents | Position |
|---|---|
| Buffer MLB Mixture + Sample | Lane A, Lane B, Lane C, Lane D: Pos 1 |
| Buffer MW1 | Lane A, Lane B, Lane C, Lane D: Pos 2 |
| Buffer MW2 | Lane A, Lane B, Lane C, Lane D: Pos 3 |
| Nuclease-Free Water | Lane A, Lane B, Lane C, Lane D: Pos 6 |

Each lane can extract one 96-well plate of samples, with full load, we can extract 384 samples in one run.
Load the Script of nucleic Acid Extraction and run the extraction.
The whole run will take approximate 20 minutes.

### Preparation of Reagents and Assay Mixes

### Preparation of Reagents

1) Thaw the primer and probe mix, Positive Control, Nuclease-Free Water and PCR Master Mix provided.
2) Thaw all reaction mixes at room temperature for a minimum of 30 minutes.
3) Keep all thawed reagents on ice.
4) Vortex all components except the PCR Master Mix and Primer and Probe Mix for 5 seconds and perform a quick spin.
5) The PCR Master Mix and Primer/probe mix should be mixed gently by inverting the tube a few times.

Prior to use, ensure that any precipitate in the PCR Master Mix is re-suspended by pipetting up and down multiple times. Do not leave kit components at room temperature for more than 2 hours. The PCR reactions are set up in a total volume of 10 μL/reaction. Table 5 shows the component volumes for each 10 μL reaction.

TABLE 5
Assay Components and Reaction Volume

| Components | Volume/Reaction | |
|---|---|---|
| 5′ PCR Master Mix | 2 | μL |
| 5′ Primer and | 2 | μL |
| Probe Mix | | |
| DNA sample or | Sample: 6 μL | |
| Controls | [symbol] s: add 2 μL of controls and add 4 μL | |
| | of nuclease-free water to make 6 μL | |
| Total Volume | 10 | μL |

[symbol]

indicates data missing or illegible when filed

For accuracy, PCR Master Mix, primers and probes should be pre-mixed into assay mixes as described in Table 6 below.

### Preparation of Assay Mixes

Assay mixes should be prepared just prior to use. Label a microcentrifuge tube (not provided) for each reaction mix, as shown in Table 6. For each control and virus detection reaction, prepare sufficient working assay mixes for the DNA samples, one Positive Control, one extraction control and one nuclease-free water for No Template Control (NTC), according to the volumes in Table 6. Include reagents for 1 extra sample to allow sufficient overage for the PCR set-up. The assay mixes contain all of the components needed for PCR except the templates (sample or controls).

TABLE 6
Preparation of Assay Mixes

| | Volume of 5′ PCR Master | Volume of 5′ Primer and probe |
|---|---|---|
| Assay | 2 μL × (n + 3 + 1) | 2 μL × ( n + 3 + 1) |
| Mix | | |

n = number of reactions (DNA samples), +3 is for 3 controls. Prepare enough for 1 extra sample (+1) to allow for sufficient coverage for the PCR set-up.

A reaction mix containing all reagents, except for the DNA sample or control templates, was prepared for the total number of samples and controls to be tested in one run. The Positive Control (PC), Extraction Control (EC) and No Template Control (NTC) should be included in each run.

### Run Layout

For each reaction, add 4 μL of the appropriate assay mix to the plate or tubes. Add up to 6 μL of template. The assay has been validated on the following PCR instruments:

TABLE 7
Validated PCR Instruments

| Company | Model |
|---|---|
| Bio-Rad | CFX384 |
| Bio-Rad | CFX96 |
| Thermo Fisher (ABI) | QuantStudio 5 |
| Thermo Fisher (ABI) | 7500 Fast Dx |
| Roche | LightCycler 480 II |

TABLE 8a
Plate Layout for 384-Well Plate

| | 1 | 3 | 5 | 7 | 9 | 11 | 13 | 15 | 17 | 19 | 21 | 23 |
|---|---|---|---|---|---|---|---|---|---|---|---|---|
| A | NTC | EC | S1 | S2 | S3 | S4 | S5 | S6 | S7 | S8 | S9 | PC |
| B | S10 | S11 | S12 | S13 | S14 | S15 | S16 | S17 | S18 | S19 | S20 | S21 |
| C | S22 | S23 | S24 | S25 | S26 | S27 | S28 | S29 | S30 | S31 | S32 | S33 |
| D | S34 | S35 | S36 | S37 | S38 | S39 | S40 | S41 | S42 | S43 | S44 | S45 |
| E | S46 | S47 | S48 | S49 | S50 | S51 | S52 | S53 | S54 | S55 | S56 | S57 |
| F | S58 | S59 | S60 | S61 | S62 | S63 | S64 | S65 | S66 | S67 | S68 | S69 |
| G | S70 | S71 | S72 | S73 | S74 | S75 | S76 | S77 | S78 | S79 | S80 | S81 |
| H | S82 | S83 | S84 | S85 | S86 | S87 | S88 | S89 | S90 | S91 | S92 | S93 |
| I | S94 | S95 | S96 | S97 | S98 | S99 | S100 | S101 | S102 | S103 | S104 | S105 |
| J | S106 | S107 | S108 | S109 | S110 | S111 | S112 | S113 | S114 | S115 | S116 | S117 |
| K | S118 | S119 | S120 | S121 | S122 | S123 | S124 | S125 | S126 | S127 | S128 | S129 |
| L | S130 | S131 | S132 | S133 | S134 | S135 | S136 | S137 | S138 | S139 | S140 | S141 |
| M | S142 | S143 | S144 | S145 | S146 | S147 | S148 | S149 | S150 | S151 | S152 | S153 |
| N | S154 | S155 | S156 | S157 | S158 | S159 | S160 | S161 | S162 | S163 | S164 | S165 |
| O | S166 | S167 | S168 | S169 | S170 | S171 | S172 | S173 | S174 | S175 | S176 | S177 |
| P | S178 | S179 | S180 | S181 | S182 | S183 | S184 | S185 | S186 | S187 | S188 | S189 |

A single experiment can analyze up to 381 unknown samples. PC, Positive Control; EC, Extraction Control; NTC, No Template Control (water); S1-S189, Samples 1-189 (up to 381 unknown samples can be loaded).

After all reagents have been added to the plate, tightly seal the plate to prevent evaporation. Spin at 1,000 rpm for 1 minute to mix and collect all the reagents at the bottom of the plate wells. Place in the real-time PCR instrument immediately.

TABLE 8b
Plate Layout for 96-well Plate

| | | 1 | 2 | 3 | 4 | 5 | 6 | 7 | 8 | 9 | 10 | 11 | 12 |
|---|---|---|---|---|---|---|---|---|---|---|---|---|---|
| A | Assay Mix | NTC | EC | S1 | S2 | S3 | S4 | S5 | S6 | S7 | S8 | S9 | PC |
| B | Assay Mix | S10 | S11 | S12 | S13 | S14 | S15 | S16 | S17 | S18 | S19 | S20 | S21 |
| C | Assay Mix | S22 | S23 | S24 | S25 | S26 | S27 | S28 | S29 | S30 | S31 | S32 | S33 |
| D | Assay Mix | S34 | S35 | S36 | S37 | S38 | S39 | S40 | S44 | S42 | S43 | S44 | S45 |
| E | Assay Mix | S46 | S47 | S48 | S49 | S50 | S55 | S52 | S53 | S54 | S55 | S56 | S57 |
| F | Assay Mix | S58 | S59 | S60 | S61 | S62 | S63 | S64 | S65 | S66 | S67 | S68 | S69 |
| G | Assay Mix | S70 | S71 | S72 | S73 | S74 | S75 | S76 | S77 | S78 | S79 | S80 | S81 |
| H | Assay Mix | S82 | S83 | S84 | S85 | S86 | S87 | S88 | S89 | S90 | S91 | S92 | S93 |

A single experiment can analyze up to 93 unknown samples. PC, Positive Control; EC, Extraction Control; NTC, No-Template Control (water); S1-S93, Samples 1-93 (up to 93 unknown samples can be loaded).

After all reagents have been added to the plate, tightly seal the plate to prevent evaporation. Spin at 1000 rpm for 1 minute to mix and collect all the reagents at the bottom of plate wells. Place in the real-time PCR instrument immediately.

### Instrument Set-Up

Set up the PCR reaction thermocycling conditions on ABI QuantStudio 5, ABI 7500 Fast Dx, or Bio-Rad CXF 384 Real-Time PCR Instrument as follows.
a) Selection of Detectors
   1. For ABI QuantStudio 5 and ABI 7500 Fast Dx, assign the target J2L as the target B6R as IC/HEX and the RNase P (Internal control) as y5 respectively.
   2. For Bio-Rad CFX 384/96, select all channel.
   3. For Roche LightCycler 480 II, in detection format select AM EX and y5.

Setup the thermocycling parameters for QuantStudio 5 (QS5) Real-Time PCR Instrument, ABI 7500 Fast Dx, BioRad CFX384/96, and Roche LightCycler 480 II as shown in Table 9a and Table 9b.

TABLE 9a
PCR Cycling Parameters on ABI QS5 and ABI 7500 Fast Dx

| | | | Ramp | | |
|---|---|---|---|---|---|
| | Temperature | Time | Rate | | Data |
| Step | (° C.) | (Seconds) | (° C./s) | Cycles | Collection |
| Polymerase | 95 | 120 | 1.6 | 1 | OFF |
| Activation | | | | | |
| Denaturation | 95 | 3 | 1 | 45 | OFF |
| Annealing | 60 | 30 | 1 | | FAM, |
| and | | | | | HEX and |
| Extension | | | | | Cy5 |

TABLE 9b
PCR Cycling Parameters on Bio-Rad
CFX 384/96 and LightCycler 480 II

| | Temperature | Time | | Data |
|---|---|---|---|---|
| Step | (° C.) | (Seconds) | Cycles | Collection |
| Polymerase | 95 | 120 | 1 | OFF |
| activation | | | | |
| Denaturation | 95 | 3 | ′45 | OFF |
| Annealing and | 60 | 30 | | FAM, |
| Extension | | | | HEX and |
| | | | | Cy5 |

### Start the Run

### DATA ANALYSIS

### Assessment of Real-Time PCR Results

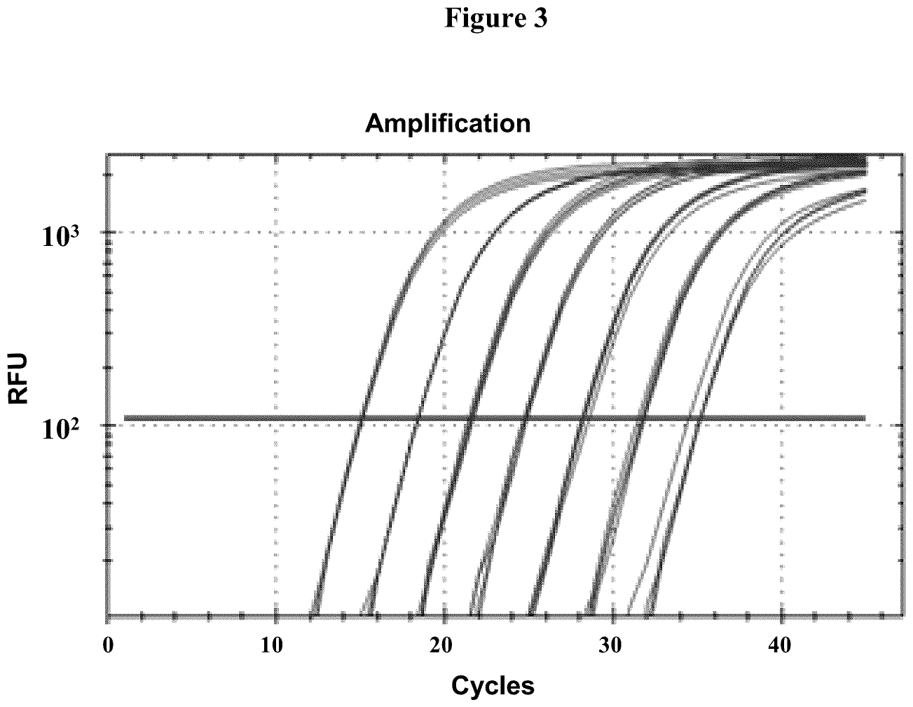

Save and analyze the data following the instrument manufacturer's instruction. Adjust the threshold above any background signal to around the middle of the exponential phase of the amplification curve in the log view (e.g., FIG. 3). The procedure chosen for setting the threshold should be used consistently. Exact threshold setting may be different for individual instruments and can be adjusted based on the amplification curves if needed.

### Assessment of the Assay Run

### ABI QuantStudio 5

### A. Cq Values for Controls

The Quanti Virus MPXV Test Kit of the invention protocol dictates that the controls should be analyzed before the analysis of patient samples. The kit positive, extraction and no template control Cq values must meet the acceptance criteria in Table 10a below for the assay to be valid. If kit control(s) fail, the test is invalid and needs to be repeated. Patient sample data is analyzed and interpreted only after all the kit controls pass.

TABLE 10a
Acceptable Cq Values for Positive Control,
Extraction Controls and No Template Control

| | | |
|---|---|---|
| | Acceptable | Test valid/ |
| Control | Cq | invalid |

| | | | |
|---|---|---|---|
| Extraction | RPP30 gene | <38 | Valid |
| control | | | |
| Positive | J2L gene | <30 | Valid |
| control | B6R gene | <30 | Valid |

| | | |
|---|---|---|
| Non-template control | ≥45 | Valid |

### A. Cq Values for Samples

Assessment of the results for each individual assay should be based on the Cq values, according to the criteria outlined in Table 10b below.

TABLE 10b
Individual Assay Results

| Target | Cut-Off | Result |
|---|---|---|
| J2L Virus Gene (MPXV- | Cq <40 | POS |
| specific) | | |
| J2L Virus Gene (MPXV- | Cq ≥40 | NEG |
| specific) | | |
| B6R Virus Gene (MPXV- | Cq <40 | POS |
| specific) | | |
| B6R Virus Gene (MPXV- | Cq ≥40 | NEG |
| specific) | | |
| Human RPP30 Gene | Cq <38 | DNA input OK |
| Human RPP30 Gene | Cq ≥38 | DNA input fail |

### ABI 7500 FAST Dx

### A. Cq Values for Controls

The Quanti Virus MPXV Test Kit of the invention protocol dictates that the controls be analyzed before the analysis of patient samples. The kit positive, extraction and no template control Cq values must meet the acceptance criteria in Table 11a below for the assay to be valid. If kit control(s) fail, the test is invalid and needs to be repeated. Patient sample data is analyzed and interpreted only after all the kit controls pass.

TABLE 11a
Acceptable Cq Values for Positive Control,
Extraction Control and No Template Control

| | | |
|---|---|---|
| | Acceptable | Test valid/ |
| Control | Cq | invalid |

| | | | |
|---|---|---|---|
| Extraction | RPP30 gene | <39 | Valid |
| Control | | | |
| Positive | J2L gene | <30 | Valid |
| Control | B6R gene | <30 | Valid |

| | | |
|---|---|---|
| No Template Control | ≥45 | Valid |

### Cq Values for Samples

Assessment of the results for each individual assay should be based on the Cq values, according to the criteria outlined in Table 11b below.

TABLE 11b
Individual Assay Results

| Target | Cut-Off | Result |
|---|---|---|
| J2L Virus Gene (MPXV- | Cq <40 | POS |
| J2L Virus Gene (MPXV- | Cq ≥40 | NEG |
| B6R Virus Gene (MPXV- | Cq <40 | POS |
| B6R Virus Gene (MPXV- | Cq ≥40 | NEG |
| Human RPP30 Gene | Cq <38 | DNA input OK |
| Human RPP30 Gene | Cq ≥40 | DNA input fail |

### Bio-Rad CFX384 and CFX96

### A. Cq Values for Control

The Quanti Virus MPXV Test Kit of the invention protocol dictates that the controls be analyzed before the analysis of patient samples. The kit positive, extraction and no template control Cq values must meet the acceptance criteria in Table 12a below for the assay to be valid. If kit control(s) fail, the test is invalid and needs to be repeated. Patient sample data is analyzed and interpreted only after all the kit controls pass.

TABLE 12a
Acceptable Cq Values for Positive Control,
Extraction Control and No Template Control

| | | |
|---|---|---|
| | Acceptable | Test valid/ |
| Control | Cq | invalid |

| | | | |
|---|---|---|---|
| Extraction | RPP30 gene | <39 | Valid |
| control | | | |
| Positive | J2L gene | <30 | Valid |
| control | B6R gene | <30 | Valid |

| | | |
|---|---|---|
| Non-template control | ≥45 | Valid |

Cq Values for Samples

Assessment of the results for each individual assay should be based on the Cq values, according to the criteria outlined in Table 12b below:

TABLE 12b
Individual Assay Results

| Target | Cut-Off | Result |
|---|---|---|
| J2L Virus Gene (MPXV- | Cq <40 | POS |
| specific) | | |
| J2L Virus Gene (MPXV- | Cq ≥40 | NEG |
| specific) | | |
| B6R Virus Gene (MPXV- | Cq <40 | POS |
| specific) | | |
| B6R Virus Gene (MPXV- | Cq ≥40 | NEG |
| specific) | | |
| Human RPP30 Gene | Cq <38 | DNA input OK |
| Human RPP30 Gene | Cq ≥38 | DNA input fail |

### Roche LightCycler 480 II

### A. Cq Values for Control

The Quanti Virus MPXV Test Kit of the invention protocol dictates that the controls be analyzed before the analysis of patient samples. The kit positive, extraction and no template control Cq values must meet the acceptance criteria in Table 13a below for the assay to be valid. If kit control(s) fail, the test is invalid and needs to be repeated. Patient sample data is analyzed and interpreted only after all the kit controls pass.

TABLE 13a
Acceptable Cq Values for Positive Control,
Extraction Control and No Template Control

| | | |
|---|---|---|
| | | Test valid/ |
| Control | Acceptable | invalid |

| | | | |
|---|---|---|---|
| Extraction | RNase P | <39 | Valid |
| Positive Control | J2L gene | <30 | Valid |
| | B6R gene | <30 | Valid |

| | | |
|---|---|---|
| No Template Control | >45 | Valid |

### Cq Values for Samples

Assessment of the results for each individual assay should be based on the Cq values, according to the criteria outlined in Table 13b below.

TABLE 13b
Individual Assay Results

| Target | Cut-Off | Result |
|---|---|---|
| J2L Virus Gene (MPXV- | Cq <40 | POS |
| specific) | | |
| J2L Virus Gene (MPXV- | Cq ≥40 | NEG |
| specific) | | |
| B6R Virus Gene (MPXV- | Cq <40 | POS |
| specific) | | |
| B6R Virus Gene (MPXV- | Cq ≥40 | NEG |
| specific) | | |
| Human RPP30 Gene | Cq <38 | DNA input OK |
| Human RPP30 Gene | Cq ≥38 | DNA input fail |

### INTERPRETATION OF RESULTS

Positive Control, Extraction Control, and No Template Control in the kit must function as outlined in Tables 10a, 11a, 12a and 13a above. If the controls do not function as required, the test is invalid. All the samples need to be retested.

When MPXV J2L, BR6 and human RPP30 genes or one of MPXV gene (J2L or BR6) and RPP30 gene were detectable, the patient sample is positive. When MPXV J2L and BR6 were not dateable, but human RPP30 gene was detectable, the patient sample was negative. When human RPP30 gene was not detectable although one or two MPXV genes were detectable, the result was invalid and repeat the test is needed (Table 14)

TABLE 14
Interpretation of the Results

| | | RPP30 | | | |
|---|---|---|---|---|---|
| J2L Gene | B6R Gene | Gene | Status | Result | Action |
| Undetected | Detected | Undetected | Invalid | Inconclusive | Repeat test one |
| Detected | Undetected | Undetected | | | more time. |
| | | | | | If the repeat result |
| | | | | | remains invalid, |
| | | | | | consider collecting |
| | | | | | new specimen. |
| Undetected | Undetected | Detected | Valid | MPXV- | Report results to |
| | | | | Negative | healthcare provider. |
| | | | | | Consider testing for |
| | | | | | other pathogens. |
| Detected | Detected | Detected | Valid | MPXV- | Report results to |
| Undetected | Detected | Detected | | Positive | healthcare provider |
| Detected | Undetected | Detected | | | and CDC. |

### Results

### A. Limit of Detection (Analytical Sensitivity)

We purchased inactivated MPXV (USA/MA001/2022) from ZeptoMetrix LLC (Cat #0810657CFHI, NY 14201). Its titration is about 1.23×108 TCID50/mL. Healthy clinical samples (negative sample in the VTM) were acquired from San Francisco Department of Public Health (SF DPH lab) and used as background diluent in preparation of the contrived samples for PreLoD and LoD study.

The stock solution was first diluted with 10 mM Tris Buffer (pH 8.0) to reach 1×106 TCID50/mL for all the following tests. All further dilutions were done with a pool of healthy clinical background prepared fresh for each test. Extraction was performed on the MGISP-NE384 automated extractor with a sample input volume of 200 μL and elution volume of 30 μL.

The 1st round of PreLoD was done by 10-folds dilution of the MPXV virus into healthy clinical background, which covers concentrations from 1×104 TCID50/mL through 1×10−3 TCID50/mL. Extraction was done on 5 replicates at each concentration, after which 6 μL elute was combined with 4 μL PCR assay mix for each reaction. The estimated LoD was determined to be in between 10 to 100 TCID50/mL (Table 15).

TABLE 15
Pre-LoD Test for QuantiVirus ™ MPXV Test

| 10,000 TCID₅₀/mL | | | | | | Avg | Std | CV |
|---|---|---|---|---|---|---|---|---|
| J2L | 27.62 | 29.04 | 27.05 | 24.91 | 28.46 | 27.41 | 1.597 | 0.058 |
| B6R | 29.38 | 30.06 | 28.07 | 26.56 | 30.10 | 28.83 | 1.513 | 0.052 |
| RP | 23.36 | 24.07 | 24.14 | 23.07 | 24.20 | 23.77 | 0.517 | 0.022 |
| 1,000 TCID₅₀/mL | | | | | | Avg | Std | CV |
| J2L | 31.33 | 32.33 | 31.72 | 31.18 | 32.21 | 31.75 | 0.514 | 0.016 |
| B6R | 34.03 | 33.18 | 33.08 | 33.19 | 32.75 | 33.24 | 0.474 | 0.014 |
| RP | 24.15 | 23.50 | 23.83 | 24.00 | 23.73 | 23.84 | 0.247 | 0.010 |
| 100 TCID₅₀/mL | | | | | | Avg | Std | CV |
| J2L | 31.98 | 37.19 | | 35.15 | 36.74 | 35.27 | 2.360 | 0.067 |
| B6R | 33.64 | | 34.49 | 34.69 | 36.05 | 34.72 | 0.996 | 0.029 |
| RP | 23.76 | 23.79 | 24.00 | 23.27 | 24.00 | 23.77 | 0.302 | 0.013 |

| 10 TCID₅₀/mL | | | | | | Avg | Std | CV |
|---|---|---|---|---|---|---|---|---|
| J2L | | | | 34.35 | | | | |
| B6R | | | | | | | | |
| RF | 23.67 | 23.43 | 24.18 | 24.00 | 24.86 | | | |
| 1 TCID₅₀/mL | | | | | | Avg | Std | CV |
| J2L | | | | | | | | |
| B6R | | | | | | | | |
| RP | 23.41 | 24.19 | 23.76 | 24.47 | 23.53 | | | |
| 0.1 TCID₅₀/mL | | | | | | Avg | Std | CV |
| J2L | | | | | | | | |
| B6R | | | | | | | | |
| RP | 23.98 | 23.78 | 23.85 | 24.19 | 24.08 | | | |
| 0.01 TCID₅₀/mL | | | | | | Avg | Std | CV |
| J2L | | | | | | | | |
| B6R | | | | | | | | |
| RP | 23.95 | 23.46 | 23.36 | 23.10 | 23.91 | | | |
| 0.001 TCID₅₀/mL | | | | | | Avg | Std | CV |
| J2L | | | | | | | | |
| B6R | | | | | | | | |
| RP | 23.32 | 23.27 | 23.41 | 23.13 | 23.42 | | | |

The 2nd round of PreLoD was done with focused dilution range that covers concentrations of 20, 40, 60, 80 and 100 TCID50/mL. Five (5) replicates were extracted and tested with qPCR assay at each concentration. Result showed proposed LoD to be around 40 to 60 TCID50/mL (Table 16).

TABLE 16
Second round Pre-LoD Test for QuantiVirus MPXV Test

| 100 TCID₅₀/mL | | | | | | Avg | Std | CV |
|---|---|---|---|---|---|---|---|---|
| J2L | 34.05 | | 37.33 | 34.21 | 32.59 | 34.55 | 1.994 | 0.058 |
| B6R | 34.81 | | 36.72 | | 35.33 | 35.62 | 0.991 | 0.028 |
| RP | 24.89 | 25.13 | 24.67 | 24.87 | 24.77 | 24.87 | 0.171 | 0.007 |
| 80 TCID₅₀/mL | | | | | | Avg | Std | CV |
| J2L | 35.56 | 40.49 | | 35.23 | 34.95 | 36.56 | 2.634 | 0.072 |
| B6R | 36.29 | 45.42 | 35.92 | 40.53 | 40.04 | 39.64 | 3.853 | 0.097 |
| RP | 25.75 | 26.27 | 25.85 | 26.23 | 25.00 | 25.82 | 0.514 | 0.020 |
| 60 TCID₅₀/mL | | | | | | Avg | Std | CV |
| J2L | 34.31 | | | 35.26 | 34.78 | 34.78 | 0.475 | 0.014 |
| B6R | 35.68 | | 36.60 | 34.33 | | 35.54 | 1.139 | 0.032 |
| RP | 24.99 | 25.03 | 25.55 | 25.02 | 24.43 | 25.00 | 0.397 | 0.016 |
| 40 TCID₅₀/mL | | | | | | Avg | Std | CV |
| J2L | | | | 34.68 | | 34.68 | N/A | N/A |
| B6R | 36.02 | 36.99 | 36.05 | 35.88 | 38.52 | 36.69 | 1.115 | 0.030 |
| RP | 25.01 | 25.99 | 26.06 | 25.16 | 24.90 | 25.42 | 0.559 | 0.022 |

| 20 TCID₅₀/mL | | | | | | Avg | Std | CV |
|---|---|---|---|---|---|---|---|---|
| J2L | | | | | 45.36 | | | |
| B6R | | | | | | | | |
| RP | 25.13 | 25.15 | 25.87 | 24.99 | 25.39 | | | |

The final test of clinical sensitivity (LoD) was done with dilutions that cover 30, 40, 50, 60, 80, 100, 150 and 200 TCID50/mL and 24 replicates at each concentration. Final LoD was confirmed as the lowest concentration that reached positive detection rate of 95% and above, and the clinical LoD of our Monkeypox Detection Assay is 100 TCID50/mL (see Table 17).

TABLE 17
LoD Test for QuantiVirus ™ MPXV Test

| 200 | | | | | | | | | | | | |
|---|---|---|---|---|---|---|---|---|---|---|---|---|
| TCID50/mL | | | | | | | | | | | | |
| J2L | 33.44 | 32.63 | 32.61 | 32.72 | 32.39 | 33.52 | 33.04 | 35.20 | 33.35 | 34.99 | 32.51 | 33.04 |
| B6R | 35.18 | 35.26 | 34.83 | 35.60 | 35.23 | 35.35 | 34.98 | 34.65 | 34.49 | 36.75 | 33.69 | 36.84 |
| RP | 26.30 | 26.25 | 26.15 | 25.71 | 25.90 | 26.30 | 26.00 | 26.12 | 26.04 | 25.45 | 25.52 | 25.62 |
| 200 | | | | | | | | | | | | |
| TCID50/mL | | | | | | | | | | | | |
| J2L | 31.80 | 32.18 | 32.33 | 34.11 | 32.02 | 32.05 | 32.67 | 31.99 | 33.42 | 33.36 | 32.23 | 32.77 |
| B6R | 34.05 | 35.48 | 35.32 | | 36.47 | 36.28 | 35.02 | 35.20 | 34.79 | 37.37 | | 35.34 |
| RP | 25.63 | 26.16 | 26.69 | 26.49 | 26.25 | 26.53 | 26.47 | 26.45 | 26.45 | 27.10 | 25.22 | 26.24 |
| 150 | | | | | | | | | | | | |
| TCID50/mL | | | | | | | | | | | | |
| J2L | 33.92 | 33.36 | 33.54 | 35.47 | 32.94 | 36.30 | 33.57 | 32.46 | 34.74 | | 33.60 | 33.10 |
| B6R | 36.72 | 36.61 | 36.54 | | 35.01 | 37.08 | 35.10 | 35.32 | 35.61 | 34.68 | 35.52 | 39.06 |
| RP | 26.59 | 26.78 | 27.10 | 27.20 | 26.74 | 27.05 | 26.44 | 26.32 | 26.54 | 26.18 | 25.65 | 26.35 |
| 150 | | | | | | | | | | | | |
| TCID50/mL | | | | | | | | | | | | |
| J2L | 33.10 | 34.66 | 32.70 | 35.31 | 33.31 | 32.68 | 33.25 | 32.59 | 33.33 | 32.09 | 35.27 | 33.12 |
| B6R | | 36.19 | 35.44 | | 35.51 | | 34.39 | 35.08 | 35.27 | 34.71 | 36.25 | 36.86 |
| RP | 26.40 | 26.56 | 26.46 | 26.59 | 26.28 | 27.04 | 28.37 | 26.41 | 26.13 | 26.30 | 26.48 | 25.87 |
| 100 | | | | | | | | | | | | |
| TCID50/mL | | | | | | | | | | | | |
| J2L | 33.33 | 34.15 | 33.08 | 34.31 | 33.39 | 33.96 | 32.55 | | 34.05 | | 35.27 | 35.80 |
| B6R | 35.99 | | 36.46 | 36.62 | | 45.78 | 35.16 | 37.06 | 38.39 | 37.50 | 36.73 | 40.31 |
| RP | 26.36 | 26.29 | 26.88 | 26.51 | 27.08 | 26.47 | 27.03 | 26.69 | 26.79 | 26.38 | 26.39 | 26.68 |
| 100 | | | | | | | | | | | | |
| TCID50/mL | | | | | | | | | | | | |
| J2L | 33.14 | 34.76 | 33.89 | 33.57 | 31.67 | 34.13 | 34.41 | 33.61 | 32.98 | 34.07 | 34.26 | 33.51 |
| B6R | 35.72 | 35.01 | | 36.45 | | 36.07 | 35.08 | | 38.63 | 36.23 | 36.25 | 36.91 |
| RP | 26.52 | 26.36 | 26.55 | 26.27 | 26.60 | 26.61 | 26.26 | 26.02 | 25.78 | 26.36 | 26.25 | 25.89 |
| 80 | | | | | | | | | | | | |
| TCID50/mL | | | | | | | | | | | | |
| J2L | 34.08 | 35.21 | 34.66 | 34.28 | 34.59 | 33.49 | 33.89 | | 34.06 | 34.57 | 32.76 | 35.34 |
| B6R | 45.58 | | 36.32 | 40.21 | 36.17 | 36.59 | | | 36.06 | 36.19 | 36.77 | 43.98 |
| RP | 26.41 | 25.67 | 26.45 | 26.41 | 26.27 | 24.82 | 26.35 | 26.28 | 26.14 | 24.66 | 26.12 | 25.31 |
| 80 | | | | | | | | | | | | |
| TCID50/mL | | | | | | | | | | | | |
| J2L | 34.05 | 33.19 | 32.96 | | 34.39 | 35.32 | 34.00 | | 33.57 | 34.02 | 33.51 | |
| B6R | 38.76 | 36.17 | 38.94 | | 34.48 | 40.78 | 35.81 | 37.84 | 39.56 | 35.78 | 36.35 | |
| RP | 26.12 | 26.29 | 26.29 | 26.21 | 25.51 | 24.80 | 26.37 | 26.51 | 26.28 | 26.22 | 26.17 | 26.06 |
| 60 | | | | | | | | | | | | |
| TCID50/mL | | | | | | | | | | | | |
| J2L | | | 33.72 | | 34.42 | 33.35 | 34.71 | | | 34.22 | 33.71 | 35.24 |
| B6R | 35.32 | 40.22 | 37.04 | | 36.24 | 39.52 | | | 36.49 | 36.14 | 36.82 | |
| RP | 25.96 | 26.37 | 26.42 | 26.53 | 26.54 | 26.14 | 26.17 | 25.54 | 26.11 | 25.80 | 26.24 | 26.14 |
| 60 | | | | | | | | | | | | |
| TCID50/mL | | | | | | | | | | | | |
| J2L | 35.17 | | 34.33 | | 33.28 | | 33.29 | 34.63 | 34.62 | 33.80 | | 35.88 |
| B6R | 35.50 | 37.33 | 35.24 | | | | 36.87 | 37.07 | | | | |
| RP | 26.17 | 26.55 | 26.82 | 26.06 | 26.40 | 25.18 | 25.70 | 26.20 | 25.23 | 25.76 | 26.01 | 25.53 |
| 50 | | | | | | | | | | | | |
| TCID50/mL | | | | | | | | | | | | |
| J2L | 34.60 | | 35.25 | | 35.37 | 31.98 | | | 35.73 | 35.00 | | 35.30 |
| B6R | 36.36 | | 45.58 | 36.35 | | | | 43.19 | | 37.18 | | 41.29 |
| RP | 26.40 | 26.23 | 26.19 | 26.48 | 26.05 | 25.76 | 26.43 | 25.94 | 26.09 | 25.88 | 25.84 | 25.65 |
| 50 | | | | | | | | | | | | |
| TCID50/mL | | | | | | | | | | | | |
| J2L | | | 33.39 | 34.27 | | 34.03 | 32.67 | | | | 34.98 | |
| B6R | | | | | | | | 37.56 | 36.25 | 36.30 | 36.01 | |
| RP | 26.35 | 26.62 | 26.39 | 26.37 | 26.20 | 27.19 | 26.58 | 27.03 | 26.39 | 26.14 | 26.25 | 23.53 |
| 40 | | | | | | | | | | | | |
| TCID50/mL | | | | | | | | | | | | |
| J2L | 35.31 | 35.57 | 35.14 | | 34.90 | 33.32 | 34.92 | | | 35.53 | 34.29 | 35.43 |
| B6R | | | | | 38.24 | 36.06 | | | | | 37.81 | 34.97 |
| RP | 26.28 | 26.76 | 26.82 | 27.36 | 27.96 | 26.47 | 27.04 | 26.81 | 26.44 | 26.96 | 26.10 | 26.13 |

| | | | | | | | | | | | |
|---|---|---|---|---|---|---|---|---|---|---|---|
| 40 | | | | | | | | | | | |
| TCID50/mL | | | | | | | | | | | |
| J2L | 35.45 | | | 33.26 | | 33.47 | 34.18 | | | 34.20 | |
| B6R | | 37.73 | 37.34 | | | | | | 37.42 | 37.06 | |
| RP | 26.77 | 27.03 | 26.73 | 26.46 | 27.17 | 26.81 | 27.04 | 28.83 | 25.45 | 26.17 | 26.12 |

| 30 | | | | | | | | | | | | |
|---|---|---|---|---|---|---|---|---|---|---|---|---|
| TCID50/mL | | | | | | | | | | | | |
| J2L | | 33.30 | | 34.48 | 34.23 | | | 40.98 | | 34.01 | 34.68 | |
| B6R | | | | | | 37.25 | | | | | | |
| RP | 26.37 | 26.03 | 26.10 | 26.37 | 26.55 | 26.33 | 26.24 | 26.25 | 26.32 | 26.36 | 26.19 | 25.67 |
| 30 | | | | | | | | | | | | |
| TCID50/mL | | | | | | | | | | | | |
| J2L | 34.71 | | | 35.47 | 35.89 | 34.47 | 34.83 | 35.26 | 35.39 | 35.69 | | |
| B6R | 40.11 | | | 45.55 | | | | | | 37.50 | | |
| RP | 26.53 | 26.34 | 26.13 | 26.36 | 25.99 | 26.14 | 26.69 | 25.66 | 26.06 | 25.63 | 22.67 | 25.84 |

| | Avg | Std | CV | Rate | Overall | Percentage |
|---|---|---|---|---|---|---|
| 200 | | | | | | |
| TCID50/mL | | | | | | |
| J2L | 32.93 | 0.885 | 0.027 | 24/24 | | |
| B6R | 35.37 | 0.901 | 0.025 | 22/24 | 24/24 | 100% |
| RP | 26.13 | 0.437 | 0.017 | | | |
| 150 | | | | | | |
| TCID50/mL | | | | | | |
| J2L | 33.67 | 1.1017 | 0.033 | 23/24 | | |
| B6R | 35.85 | 1.093 | 0.030 | 20/24 | 24/24 | 100% |
| RP | 26.58 | 0.530 | 0.020 | | | |
| 100 | | | | | | |
| TCID50/mL | | | | | | |
| J2L | 33.81 | 0.89 | 0.026 | 22/24 | | |
| B6R | 37.18 | 2.459 | 0.066 | 17/24 | 24/24 | 100% |
| RP | 26.46 | 0.318 | 0.012 | | | |
| 80 | | | | | | |
| TCID50/mL | | | | | | |
| J2L | 34.10 | 0.72993 | 0.021 | 20/24 | | |
| B6R | 38.02 | 2.931 | 0.077 | 15/24 | 21/24 | 87.50% |
| RP | 25.99 | 0.555 | 0.021 | | | |
| 60 | | | | | | |
| TCID50/mL | | | | | | |
| J2L | 34.29 | 0.77563 | 0.023 | 15/24 | | |
| B6R | 36.91 | 1.486 | 0.040 | 12/24 | 18/24 | 75.00% |
| RP | 26.07 | 0.418 | 0.016 | | | |
| 50 | | | | | | |
| TCID50/mL | | | | | | |
| J2L | 34.38 | 1.16597 | 0.034 | 12/24 | | |
| B6R | 38.61 | 3.462 | 0.090 | 07/24 | 16/24 | 66.67% |
| RP | 26.17 | 0.667 | 0.025 | | | |
| 40 | | | | | | |
| TCID50/mL | | | | | | |
| J2L | 34.61 | 0.82298 | 0.024 | 15/24 | | |
| B6R | 36.97 | 1.047 | 0.028 | 9/24 | 18/24 | 75.00% |
| RP | 26.75 | 0.678 | 0.025 | | | |
| 30 | | | | | | |
| TCID50/mL | | | | | | |
| J2L | 35.24 | 1.79339 | 0.051 | 13/24 | | |
| B6R | 40.10 | 3.854 | 0.096 | 02/24 | 14/24 | 58.33% |
| RP | 26.03 | 0.769 | 0.030 | | | |

### Cross-Reactivity/Microbial Interference

### Exclusivity/Cross-Reactivity

### In Silico Analysis for Cross-Reactivity

For the MPXV primer and probe sequences, applicant evaluated sequences from 10 viruses (9 Orthopoxvirus and 1 Molluscipoxvirus). These data support that cross-reactivity is not predicted for the viruses evaluated. Results are presented in Table 18.

TABLE 18
Results of In Silico Exclusivity Analysis of MPXV primer and probe vs viral sequences

| | | | | Sequences | Sequences | |
|---|---|---|---|---|---|---|
| | | | | with >85% | with <80% | Sequences |
| | | | Number of | match to | match to at | with >80% |
| | | Upper Level | sequences | both | least one | match to |
| Species | Taxid | (Genus) | evaluated | primers | primer | probe |
| Vaccinia virus | 10245 | Orthopoxvirus | 15 | 15 | 0 | 0 |
| Cowpox Virus | 10243 | Orthopoxvirus | 27 | 25 | 2 | 0 |
| Variola virus | 10255 | Orthopoxvirus | 29 | 22 | 7 | 0 |
| (Smallpox) | | | | | | |
| Camelpox virus | 28873 | Orthopoxvirus | 21 | 18 | 3 | 0 |
| Ectromelia virus | 12643 | Orthopoxvirus | 22 | 4 | 18 | 0 |
| Uasin Gishu | 397342 | Orthopoxvirus | 3 | 2 | 1 | 0 |
| disease virus | | | | | | |
| (horsepox) | | | | | | |
| Raccoon poxvirus | 10256 | Orthopoxvirus | 1 | 0 | 1 | 0 |
| Volepox virus | 28874 | Orthopoxvirus | 15 | 2 | 13 | 0 |
| Skunkpox Virus | 160796 | Orthopoxvirus | 18 | 0 | 18 | 0 |
| Molluscum | 10279 | Molluscipoxvirus | 34 | 0 | 34 | 0 |
| Contagiosum virus | | | | | | |

### In Silico Exclusivity Analysis of MPXV PRIMERS/PROBE AGAINST BACTERIAL and Fungal Sequences

We conducted an in silico analysis for the MPXV primer/probe sequences against non-viral sequences from nine bacterial species and two fungal species that were not evaluated by wet-testing in the cross-reactivity study.

Of the non-viral sequences evaluated, no sequences demonstrated >80% homology with both MPXV primers, however, some sequences did have binding sites for the MPXV probe. We do not expect that there is any cross reactivity for these organisms due to no >80% homology in its primers although it has some probe binding sites. Results from the analysis demonstrated that for the 11 microorganisms evaluated, cross-reactivity is not predicted for the MPXV primers/probe included in the Quanti Virus MPXV Test assay. Results are presented in Table 19.

TABLE 19
Results of In Silico Exclusivity Analysis of MPXV Primer and Probe vs Non-Viral Sequences

| | | | | | Sequences | Sequences | Sequences |
|---|---|---|---|---|---|---|---|
| | | | | | with >85% | with <80% | with >80% |
| | | | | Number of | match to | match to at | match to at |
| | | | Upper level | sequences | both | least one | least for |
| Type | Species | Taxid | (Genus) | evaluated | primers | primer | one probe |
| Bacteria | Streptococcus | 1311 | Streptococcus | 239 | 0 | 239 | 0 |
| | agalactiae | | | | | | |
| Bacteria | Streptococcus | 28037 | Streptococcus | 429 | 0 | 429 | 0 |
| | mitis | | | | | | |
| Bacteria | Pseudomonas | 287 | Pseudomonas | 610 | 0 | 545 | 65 |
| | aeruginosa | | | | | | |
| Bacteria | Corynebacterium | 38289 | Corynebacterium | 42 | 0 | 42 | 0 |
| | jeikeium | | | | | | |
| Bacteria | Escherichia | 562 | Escherichia | 1000 | 0 | 975 | 25 |
| | coli | | | | | | |
| Bacteria | Acinetobacter | 471 | Acinetobacter | 688 | 0 | 684 | 4 |
| | calcoaceticus | | | | | | |
| Bacteria | Bacillus fragilis | 817 | Bacillus | 102 | 0 | 99 | 3 |
| Bacteria | Enterobacter | 1351 | Enterobacter | 710 | 0 | 669 | 41 |
| | faecalis | | | | | | |
| Bacteria | Lactobacillus | 1578 | Lactobacillus | 260 | 0 | 254 | 6 |
| | species | | | | | | |
| Fungi | Trichophyton | 5551 | Trichophyton | 2039 | 0 | 2036 | 3 |
| | rubrum | | | | | | |
| Fungi | Candida albicans | 5476 | Candida | 723 | 0 | 722 | 1 |

Exclusivity/Cross-Reactivity (Wet-Testing):

The Quanti Virus™ MPXV Test was evaluated for potential cross-reactivity with 14 commercially available microorganisms and viruses at sample concentrations of greater than 1×106 CFU/mL or copies/mL. The microorganisms or viruses were spiked at high concentrations into pooled UTM from negative lesion swab samples. No cross□reactivity was observed with any of the fourteen microorganisms and viruses evaluated in the study. Results are presented in Table 20.

TABLE 20
Cross-Reactivity

| | | | J2L | B6R | RPP30 |
|---|---|---|---|---|---|
| Organism | Concentration | | (Cq) | (Cq) | (Cq) |
| Staphylococcus | 1.28 × 10⁸ | cfu/mL | 45.0 | 45.0 | 24.5 |
| epidermidis | | | | | |
| Staphylococcus | 1.32 × 10⁹ | cfu/mL | 45.0 | 45.0 | 24.2 |
| aureus | | | | | |
| Streptococcus | 1.28 × 10⁸ | cfu/mL | 45.0 | 45.0 | 24.4 |
| pyogenes | | | | | |
| Human herpesvirus 7 | 1.0 × 10⁵ | cfu/mL | 45.0 | 45.0 | 25.0 |
| Human herpesvirus 8 | 6.0 × 10⁷ | cp/mL | 45.0 | 45.0 | 24.3 |
| Coxsackie A16 | 1.0 × 10⁵ | cp/mL | 45.0 | 45.0 | 24.4 |
| Measles | 2.3 × 10¹⁰ | cp/mL | 45.0 | 45.0 | 25.0 |
| Varicella Zoster Virus | 1.0 × 10⁵ | cp/mL | 45.0 | 45.0 | 24.9 |
| JC polyomavirus | 1.3 × 10⁸ | cp/mL | 45.0 | 45.0 | 25.3 |
| Epstein Barr Virus | 1.0 × 10⁵ | cp/mL | 45.0 | 45.0 | 24.5 |
| Human herpesvirus 1 | 9.4 × 10¹⁰ | cp/mL | 45.0 | 45.0 | 24.6 |
| Human herpesvirus 2 | 2.8 × 10⁶ | TCID₅₀/mL | 45.0 | 45.0 | 24.5 |
| Human herpesvirus 5 | 1.7 × 10⁸ | cp/mL | 45.0 | 45.0 | 25.0 |
| Treponema pallidum | 4.0 × 10⁵ | cfu/mL | 45.0 | 45.0 | 24.2 |
| Vaccinia virus | 10⁵ | copies/mL | 45.0 | 45.0 | n.a. |
| Variola virus | 10⁵ | copies/mL | 45.0 | 45.0 | n.a. |
| Cowpox virus | 10⁵ | copies/mL | 45.0 | 45.0 | n.a. |
| MPXV (ATCC) | 10⁵ | copies/mL | 21.8 | 22.0 | n.a. |

### Inclusivity/Analytical Reactivity

### Inclusivity, In Silico Analysis for MPXV Targets

An in silico inclusivity analysis was conducted by aligning the MPXV target primer and probe sequence against available monkeypox virus sequence from GenBank at NCBI database as of Sep. 12, 2022. A total of 1,099 Monkeypox virus isolation sequence was analyzed, and sequence identified was 92%-100% for both primer and probe (Table 21).

TABLE 21
Inclusivity of MPXV target

| | Number of | Sequence with >90% match to |
|---|---|---|
| Species | Sequence evaluated | both MPXV primers and probe |
| Monkeypox virus | 1,099 | 1,099 |

### Collection Media Equivalency for Any Sample Collection Media not Used in our Clinical Study

Not applicable.

### Interference Substances Study (Exogenous)

A study was performed to evaluate the impact of potentially interfering substances on the performance of the QuantiVirus MPXV Test. Before DNA extraction, the following substances were spiked in MPXV-negative clinical samples in either the presence or the absence of contrived MPXV reference material. The interfering substances study demonstrated that these interferents at the concentration indicated did not have a significant impact on the performance of the Quanti Virus MPXV Test (Table 22).

TABLE 22
Interfering Substances

| | Without MPXV reference | | | With MPXV reference | | |
|---|---|---|---|---|---|---|
| | material | | | material (3× LoD) | | |
| | J2L | B6R | RPP30 | J2L | B6R | RPP30 |
| Substance | (Cq) | (Cq) | (Cq) | (Cq) | (Cq) | (Cq) |
| Abreva (7%) | 45.0 | 45.0 | 24.7 | 33.6 | 35.8 | 27.3 |
| Acyclovir (7 mg/mL) | 45.0 | 45.0 | 25.7 | 33.9 | 36.0 | 27.9 |
| Albumin (2.2 mg/mL) | 45.0 | 45.0 | 24.5 | 35.1 | 36.6 | 26.7 |
| Mucin (60 μg/mL) | 45.0 | 45.0 | 24.5 | 33.7 | 35.4 | 26.9 |
| Hydrocortisone Cream | 45.0 | 45.0 | 25.2 | 33.3 | 35.4 | 28.2 |
| (7%) | | | | | | |
| Benadryl Cream (7%) | 45.0 | 45.0 | 24.8 | 30.0 | 31.9 | 27.3 |
| Carmex (7%) | 45.0 | 45.0 | 24.8 | 32.6 | 35.1 | 27.8 |
| Casein (7 mg/mL) | 45.0 | 45.0 | 24.0 | 33.5 | 45.0 | 26.6 |
| Lanacane (3.5%) | 45.0 | 45.0 | 24.2 | 33.5 | 35.2 | 27.1 |
| KY Jelly (7%) | 45.0 | 45.0 | 25.4 | 32.4 | 33.4 | 28.1 |
| Douche (7%) | 45.0 | 45.0 | 24.7 | 34.4 | 34.9 | 27.3 |
| Neosporin (3%) | 45.0 | 45.0 | 24.2 | 34.1 | 36.1 | 27.7 |
| Urine (7%) | 45.0 | 45.0 | 25.2 | 34.2 | 45.0 | 28.2 |
| Zine Oxide Ointment (7%) | 45.0 | 45.0 | 24.7 | 34.2 | 35.6 | 28.2 |
| Vagisil Cream (1%) | 45.0 | 45.0 | 24.5 | 33.2 | 33.5 | 27.3 |
| Cornstarch (2.5 mg/mL) | 45.0 | 45.0 | 24.6 | 34.3 | 35.7 | 27.8 |

### Clinical Sample Stability

Heat-inactivated monkeypox virus (hMPXV/USA/MA001/2022) was purchased from ZeptoMetrix LLC (Cat #0810657CFHI). The virus was originally isolated from a human in Massachusetts, USA in May of 2022 and obtained through BEI Resources. The titer of the stock solution was determined by endpoint dilution assay and confirmed to be 1.23×108 TCID50/mL by ZeptoMetrix.

Negative clinical matrix was created through a pool of healthy clinical samples and used as background diluent in preparation of the contrived samples for the sample stability study. We tested the samples which stored at room temperature for 0, 6 and 24 hrs. (Table 23a-c).

TABLE 23a
Lesion Swab samples stability in UTM for 0 hr.

| | Target | 1 | 2 | 3 | 4 | 5 | 6 | 7 | 8 | 9 | 10 | Avg | Std | CV |
|---|---|---|---|---|---|---|---|---|---|---|---|---|---|---|
| 5× | J2L | 34.00 | 33.72 | 33.95 | 33.85 | 33.16 | 33.39 | 34.05 | 33.20 | 33.61 | 32.77 | 33.57 | 0.426 | 0.013 |
| LoD | B6R | 35.17 | 35.06 | 35.05 | 35.15 | 35.07 | 35.04 | 35.39 | 34.94 | 34.98 | 34.20 | 35.01 | 0.308 | 0.009 |
| | RP | 29.42 | 29.19 | 29.20 | 29.57 | 29.06 | 28.68 | 28.19 | 28.94 | 28.73 | 28.99 | 29.00 | 0.396 | 0.014 |
| 2× | J2L | 35.19 | 35.63 | 34.49 | 35.30 | 34.36 | 34.97 | 33.64 | 33.93 | 34.82 | 34.27 | 34.66 | 0.631 | 0.018 |
| LoD | B6R | 35.11 | 39.48 | 36.39 | 36.18 | 34.75 | 37.01 | 35.20 | 34.84 | 35.15 | 36.51 | 36.06 | 1.439 | 0.040 |
| | RP | 29.20 | 29.02 | 29.34 | 28.72 | 29.12 | 28.92 | 28.81 | 29.09 | 28.64 | 28.45 | 28.93 | 0.275 | 0.009 |
| | J2L | 35.01 | 34.27 | 35.74 | 35.93 | 35.80 | 35.02 | 35.14 | 35.78 | 34.18 | 35.45 | 35.23 | 0.628 | 0.018 |
| | B6R | 38.76 | 38.52 | 35.83 | 35.46 | 36.22 | 35.99 | 39.70 | 39.83 | 36.28 | 35.26 | 37.18 | 1.803 | 0.048 |
| | RP | 29.34 | 29.33 | 29.34 | 28.88 | 29.21 | 29.09 | 29.06 | 28.71 | 28.61 | 28.57 | 29.01 | 0.304 | 0.010 |
| | J2L | 33.90 | 35.28 | 35.08 | 35.42 | 35.20 | 45.00 | 45.00 | 34.79 | 33.26 | 34.61 | 36.75 | 4.396 | 0.120 |
| | B6R | 35.72 | 39.43 | 39.15 | 35.98 | 36.13 | 37.23 | 45.00 | 35.39 | 35.20 | 35.12 | 37.43 | 3.082 | 0.082 |
| | RP | 28.75 | 29.28 | 29.03 | 28.62 | 28.92 | 28.83 | 29.16 | 28.19 | 28.44 | 28.66 | 28.79 | 0.331 | 0.012 |
| Neg | J2L | 45 | 45 | 45 | 45 | 45 | 45 | 45 | 45 | 45 | 45 | 45.00 | 0.000 | 0.000 |
| | B6R | 45 | 45 | 45 | 45 | 45 | 45 | 45 | 45 | 45 | 45 | 45.00 | 0.000 | 0.000 |
| | RP | 28.18 | 28.11 | 28.06 | 27.87 | 27.79 | 27.65 | 28.11 | 27.52 | 27.58 | 28.01 | 27.89 | 0.242 | 0.009 |

TABLE 23b
Lesion Swab samples stability in UTM for 6 hrs

| | Target | 1 | 2 | 3 | 4 | 5 | 6 | 7 | 8 | 9 | 10 | Avg | Std | CV |
|---|---|---|---|---|---|---|---|---|---|---|---|---|---|---|
| 5× | J2L | 32.60 | 34.21 | 32.59 | 33.27 | 33.07 | 34.76 | 33.67 | 33.32 | 32.58 | 33.03 | 33.31 | 0.726 | 0.022 |
| LoD | B6R | 34.29 | 34.97 | 35.18 | 34.11 | 34.01 | 34.53 | 36.31 | 35.62 | 33.60 | 34.16 | 34.68 | 0.835 | 0.024 |
| | RP | 29.09 | 29.16 | 28.73 | 28.97 | 28.88 | 28.18 | 28.80 | 28.70 | 28.31 | 28.70 | 28.75 | 0.311 | 0.011 |
| 2× | J2L | 34.95 | 34.71 | 35.63 | 36.22 | 35.31 | 33.90 | 35.35 | 34.38 | 34.36 | 35.22 | 35.00 | 0.686 | 0.020 |
| LoD | B6R | 36.25 | 35.70 | 35.58 | 36.59 | 35.15 | 34.40 | | 36.47 | 34.96 | 35.24 | 35.59 | 0.737 | 0.021 |
| | RP | 28.54 | 27.83 | 28.72 | 28.73 | 28.74 | 28.49 | 28.66 | 28.43 | 28.00 | 28.27 | 28.44 | 0.316 | 0.011 |
| | J2L | 34.64 | 34.91 | 32.75 | 33.88 | 32.45 | 32.91 | 34.85 | 34.33 | 34.06 | 33.16 | 33.79 | 0.916 | 0.027 |
| | B6R | 36.02 | 35.45 | 33.05 | 34.51 | 34.39 | 35.24 | | 35.19 | | 36.10 | 34.99 | 0.999 | 0.029 |
| | RP | 28.60 | 28.71 | 28.77 | 28.59 | 28.59 | 28.48 | 28.36 | 28.63 | 28.32 | 28.31 | 28.54 | 0.161 | 0.006 |
| | J2L | 33.80 | 34.71 | 33.33 | 34.22 | 35.88 | 34.12 | 34.20 | 30.67 | 36.12 | 34.49 | 34.15 | 1.500 | 0.044 |
| | B6R | 35.15 | | 35.49 | 38.73 | 35.52 | 35.21 | 36.03 | 33.01 | 35.21 | 35.43 | 35.53 | 1.466 | 0.041 |
| | RP | 28.68 | 28.73 | 28.83 | 29.11 | 28.83 | 28.52 | 28.35 | 28.44 | 28.60 | 27.27 | 28.53 | 0.496 | 0.017 |
| Neg | J2L | 45 | 45 | 45 | 45 | 45 | 45 | 45 | 45 | 45 | 45 | 45.00 | 0.000 | 0.000 |
| | B6R | 45 | 45 | 45 | 45 | 45 | 45 | 45 | 45 | 45 | 45 | 45.00 | 0.000 | 0.000 |
| | RP | 27.44 | 27.62 | 27.67 | 27.55 | 27.55 | 27.42 | 27.06 | 27.23 | 27.39 | 27.34 | 27.43 | 0.187 | 0.007 |

TABLE 23c
Lesion Swab samples stability in UTM for 24 hrs

| | Target | 1 | 2 | 3 | 4 | 5 | 6 | 7 | 8 | 9 | 10 | Avg | Std | CV |
|---|---|---|---|---|---|---|---|---|---|---|---|---|---|---|
| 5× | J2L | 33.24 | 33.83 | 32.42 | 32.48 | 33.51 | 32.98 | 33.00 | 33.17 | 32.40 | 31.44 | 32.85 | 0.683 | 0.021 |
| LoD | B6R | 33.75 | 33.46 | 32.52 | 33.52 | 34.56 | 34.34 | 33.50 | 34.36 | 34.55 | 33.50 | 33.81 | 0.648 | 0.019 |
| | RP | 29.00 | 28.77 | 28.77 | 28.35 | 28.80 | 28.85 | 28.88 | 28.36 | 28.51 | 28.63 | 28.69 | 0.220 | 0.008 |
| 2× | J2L | 34.21 | 45 | 33.27 | 35.57 | 33.61 | 35.05 | 34.00 | 33.51 | 34.57 | 33.14 | 34.10 | 0.831 | 0.024 |
| LoD | B6R | 35.09 | 45 | 36.37 | 34.51 | 33.61 | 35.78 | 35.06 | 34.83 | 37.43 | 36.36 | 35.45 | 1.154 | 0.033 |
| | RP | 28.31 | 28.45 | 28.00 | 28.48 | 28.46 | 28.36 | 28.09 | 28.76 | 28.48 | 28.01 | 28.34 | 0.242 | 0.009 |
| | J2L | 34.56 | 33.18 | 34.04 | 33.36 | 33.96 | 33.49 | 34.07 | 34.19 | 33.29 | 35.79 | 33.99 | 0.773 | 0.023 |
| | B6R | 37.36 | 34.85 | 36.25 | 34.84 | 36.64 | 36.35 | 33.71 | 34.38 | 34.39 | 35.09 | 35.39 | 1.185 | 0.033 |
| | RP | 28.21 | 28.44 | 28.59 | 28.36 | 28.54 | 28.48 | 28.47 | 28.59 | 27.94 | 28.20 | 28.38 | 0.207 | 0.007 |
| | J2L | 34.02 | 35.92 | | 33.99 | 34.63 | 34.08 | 35.00 | 33.89 | 34.70 | 36.03 | 34.69 | 0.818 | 0.024 |
| | B6R | 34.30 | 35.00 | 34.99 | 35.59 | 35.97 | 35.62 | 34.52 | 34.68 | 35.98 | 36.24 | 35.29 | 0.681 | 0.019 |
| | RP | 28.43 | 28.41 | 28.41 | 28.38 | 28.67 | 28.49 | 28.54 | 28.58 | 28.69 | 28.45 | 28.51 | 0.112 | 0.004 |
| Neg | J2L | 45 | 45 | 45 | 45 | 45 | 45 | 45 | 45 | 45 | 45 | 45 | | |
| | B6R | 45 | 45 | 45 | 45 | 45 | 45 | 45 | 45 | 45 | 45 | 45 | | |
| | RP | 25.80 | 28.20 | 27.72 | 27.79 | 27.42 | 28.03 | 27.95 | 27.81 | 27.59 | 27.91 | 27.62 | 0.677 | 0.025 |

### Reproducibility and Repeatability

Precision studies include intra-run, inter-run, instrument and operator variability evaluation. The assay precision was assessed by the repeated testing of samples with three different template concentrations.

Inter-assay % CV was established for same lot of reagents tested on the same instrument by the same user.

Intra-assay % CV was established through performance of kit on reference samples run in replicates of ten.

Operator variability was evaluated with one lot of reagents by two operators.

Reproducibility is demonstrated based on % CV of Ct values.

### Inter-Assay Reproducibility

In this example, the same high and low MPXV controls are run in duplicate on 12 different tubes to monitor tube-to-tube variation. The tube means for high and low are calculated and then used to calculate the overall mean, standard deviation, and % CV. Overall % CV=SD of tube means÷mean of tube means×100. The average of the high and low % CV is reported as the inter-assay CV (Table 24). The Inter-assay overall CV was <4% for this assay.

TABLE 24
Inter-Assay reproducibility

| | QuantiVirus MPXV Test | | | | | | Mean | | |
|---|---|---|---|---|---|---|---|---|---|
| | J2L | | B6R | | RP | | J2L | BR6 | RP |
| 200 | | | | | | | | | |
| TCID50/mL | | | | | | | | | |
| 1 | 33.44 | 32.61 | 35.18 | 34.83 | 26.30 | 26.15 | 33.02 | 35.01 | 26.23 |
| 2 | 32.63 | 32.72 | 35.26 | 35.60 | 26.25 | 25.71 | 32.67 | 35.43 | 25.98 |
| 3 | 32.39 | 33.04 | 35.23 | 34.98 | 25.90 | 26.00 | 32.72 | 35.10 | 25.95 |
| 4 | 33.52 | 35.20 | 35.35 | 34.65 | 26.30 | 26.12 | 34.36 | 35.00 | 26.21 |
| 5 | 33.35 | 32.51 | 34.49 | 33.69 | 26.04 | 25.52 | 32.93 | 34.09 | 25.78 |
| 6 | 34.99 | 33.04 | 36.75 | 36.84 | 25.45 | 25.62 | 34.02 | 36.79 | 25.54 |
| 7 | 31.80 | 32.33 | 34.05 | 35.32 | 25.63 | 26.69 | 32.07 | 34.68 | 26.16 |
| 8 | 32.18 | 34.11 | 35.48 | 35.9 | 26.16 | 26.49 | 33.14 | 35.48 | 26.32 |
| 9 | 32.02 | 32.67 | 36.47 | 26.47 | 26.25 | 26.47 | 32.34 | 31.47 | 26.36 |
| 10 | 32.05 | 31.99 | 36.28 | 26.45 | 26.53 | 26.45 | 32.02 | 31.37 | 26.49 |
| 11 | 33.42 | 32.23 | 34.79 | 34.90 | 26.45 | 25.22 | 32.83 | 34.84 | 25.83 |
| 12 | 33.36 | 32.77 | 37.37 | 35.34 | 27.10 | 26.24 | 33.06 | 36.35 | 26.67 |
| Mean of Means | | | | | | | 32.93 | 34.63 | 26.13 |
| SD of Means | | | | | | | 0.70 | 1.66 | 0.32 |
| CV (%) of | | | | | | | 2% | 5% | 1% |
| Means | | | | | | | | | |
| 150 | | | | | | | | | |
| TCID50/mL | | | | | | | | | |
| 1 | 33.92 | 33.10 | 36.72 | 35.90 | 26.59 | 26.40 | 33.51 | 36.31 | 26.49 |
| 2 | 33.36 | 34.66 | 36.61 | 36.19 | 26.78 | 26.56 | 34.01 | 36.40 | 26.67 |
| 3 | 33.54 | 32.70 | 36.54 | 35.44 | 27.10 | 26.46 | 33.12 | 35.99 | 26.78 |
| 4 | 35.47 | 35.31 | 35.70 | 36.00 | 27.20 | 26.59 | 35.39 | 35.85 | 26.89 |
| 5 | 32.94 | 33.31 | 35.01 | 35.51 | 26.74 | 26.28 | 33.12 | 35.26 | 26.51 |
| 6 | 36.30 | 32.68 | 37.08 | 36.90 | 27.05 | 27.04 | 34.49 | 36.99 | 27.05 |
| 7 | 33.57 | 33.25 | 35.10 | 34.39 | 26.44 | 28.37 | 33.41 | 34.74 | 27.40 |
| 8 | 32.46 | 32.59 | 35.32 | 35.08 | 26.32 | 26.41 | 32.52 | 35.20 | 26.36 |
| 9 | 34.74 | 33.33 | 35.61 | 35.27 | 26.54 | 26.13 | 34.04 | 35.44 | 26.34 |
| 10 | 32.50 | 32.09 | 34.68 | 34.71 | 26.18 | 26.30 | 32.29 | 34.69 | 26.24 |
| 11 | 33.60 | 35.27 | 35.52 | 36.25 | 25.65 | 26.48 | 34.44 | 35.89 | 26.07 |
| 12 | 33.10 | 33.12 | 39.06 | 36.86 | 26.35 | 25.87 | 33.11 | 37.96 | 26.11 |
| Mean of Means | | | | | | | 33.62 | 35.89 | 26.58 |
| SD of Means | | | | | | | 0.89 | 0.94 | 0.40 |
| CV (%) of | | | | | | | 3% | 3% | 2% |
| Means | | | | | | | | | |
| Inter-Assay CV | | | | | | | 2.4% | 3.7% | 1.4% |
| (%) | | | | | | | | | |

### Intra-Assay Reproductivity

Each assay at three sample template concentrations was repeated 10 times and run on the sample plate. Average Ct and CV were calculated (Table 25).

TABLE 25
Intra assay of the target for MPXV detection kit

| | | | | MPXV B6R Gene (HEX) | | | Reference RP (Cy5) | | |
|---|---|---|---|---|---|---|---|---|---|
| MPXV | MPXV J2L Gene (FAM) | | | | | Coefficient | | | Coefficient |
| Sample | | Replicate | Coefficient | | Replicate | of | | Replicate | of |
| concentration | Mean | Detection | of | Mean | Detection | Variation | Mean | Detection | Variation |
| TCID₅₀/mL | Cq | (%) | Variation | Cq | (%) | (%) | Cq | (%) | (%) |
| 200 | 32.63 | 100 | 1.66% | 34.98 | 100 | 2.28% | 26.02 | 100 | 1.19% |
| 150 | 33.92 | 100 | 2.30% | 35.70 | 80 | 2.15% | 26.71 | 100 | 2.70% |
| 100 | 33.66 | 100 | 3.26% | 36.13 | 90 | 2.98% | 26.57 | 100 | 0.96% |

The Intra assay overall CV was <3.5% for this assay

### Operator Reproducibility

The assay reactions were set up by two operators using the same lot of reagents and run on the same instrument. Average Ct and CV were calculated (Table 26). Overall CV for two operators is <3% for this assay.

TABLE 26
Different Operator Reproducibility

| | MPXV | | | | | | | | | |
|---|---|---|---|---|---|---|---|---|---|---|
| | Sample | | | | | | | | | |
| Assay | concentration | Operator 1 | | | Operator 2 | | | Overall | | |
| Target | (TCID50/mL) | Avg | Std | CV | Avg | Std | CV | Avg | Std | CV |
| J2L | 200 | 33.29 | 1.02 | 3.05% | 32.66 | 0.75 | 2.31% | 32.98 | 0.93 | 2.81% |
| Gene | 150 | 33.73 | 0.91 | 2.68% | 33.35 | 0.96 | 2.88% | 33.53 | 0.93 | 2.77% |
| | 100 | 33.77 | 0.85 | 2.52% | 33.56 | 0.86 | 2.56% | 33.65 | 0.84 | 2.48% |
| B6R | 200 | 35.06 | 0.79 | 2.26% | 35.45 | 0.96 | 2.71% | 35.25 | 0.87 | 2.48% |
| Gene | 150 | 35.68 | 0.76 | 2.13% | 35.43 | 0.79 | 2.23% | 35.56 | 0.76 | 2.14% |
| | 100 | 36.74 | 0.97 | 2.63% | 36.29 | 1.25 | 3.44% | 36.53 | 1.09 | 2.98% |
| RP | 200 | 25.95 | 0.30 | 1.14% | 26.39 | 0.38 | 1.43% | 26.17 | 0.40 | 1.54% |
| Gene | 150 | 26.55 | 0.45 | 1.69% | 26.54 | 0.68 | 2.55% | 26.55 | 0.56 | 2.11% |
| | 100 | 26.64 | 0.29 | 1.10% | 26.26 | 0.28 | 1.07% | 26.45 | 0.34 | 1.29% |

### Intra-Instrument Reproducibility

Assay reactions were set up with 12 replicates and run on 5 different qPCR instruments including BioRad CFX 384, ABI QS 5, Roche LC 480 II, ABI 7500 Fast Dx and BioRad CFX 96. Average Ct and CV were calculated. The results indicate that five instruments have <5% CV and is acceptable.

TABLE 27
Intra-instrument Reproducibility

| | MPXV | | | | | | | | | |
|---|---|---|---|---|---|---|---|---|---|---|
| | Sample | BioRad CFX 384 | | | ABI QS 5 | | | Roche LC 480 II | | |
| | concentration | AVE | | | AVE | | | AVE | | |
| Target | copies/ml | Cq | SD | CV | Cq | SD | CV | Cq | SD | CV |
| | 40 | 33.48 | 0.38 | 1.13% | 34.09 | 0.65 | 1.90% | 34.29 | 0.8 | 2.27% |
| MPXV J2L Gene | 30 | 34.99 | 1.02 | 2.92% | 34.72 | 0.67 | 1.94% | 34.98 | 0.5 | 1.45% |
| | 25 | 34.19 | 0.8 | 2.34% | 35.27 | 0.72 | 2.05% | 34.88 | 0.8 | 2.27% |
| | 40 | 35.65 | 0.5 | 1.41% | 37.34 | 0.98 | 2.64% | 35.63 | 1.1 | 3.02% |
| MPXV B6R Gene | 30 | 36.86 | 1.05 | 2.85% | 38.08 | 0.83 | 2.17% | 36.28 | 0.8 | 2.28% |
| | 25 | 36.46 | 0.96 | 2.63% | 38.12 | 0.71 | 1.87% | 36.43 | 1 | 2.60% |
| | 40 | 28.11 | 0.21 | 0.73% | 28.11 | 0.37 | 1.30% | 29.19 | 0.2 | 0.63% |
| Reference RP | 30 | 28.39 | 0.26 | 0.92% | 28.35 | 0.24 | 0.86% | 29.7 | 0.2 | 0.50% |
| | 25 | 28.49 | 0.19 | 0.68% | 28.56 | 0.27 | 0.94% | 29.8 | 0.3 | 0.85% |

| | ABI 7500 Fast DX | | | BioRad CFX 96 | | |
|---|---|---|---|---|---|---|
| | AVE | | | AVE | | |
| Target | Cq | SD | CV | Cq | SD | CV |
| | 33.63 | 0.53 | 1.58% | 34.47 | 0.89 | 2.58% |
| MPXV J2L Gene | 33.81 | 1.14 | 3.37% | 35.07 | 0.75 | 2.14% |
| | 34.85 | 1.1 | 3.16% | 35.11 | 0.92 | 2.62% |
| | 36.03 | 1.42 | 3.93% | 37.38 | 1.27 | 3.40% |
| MPXV B6R Gene | 36.57 | 1.31 | 3.59% | 36.86 | 0.5 | 1.36% |
| | 36.74 | 1.7 | 4.63% | 37.09 | 0.9 | 2.44% |
| | 24.91 | 0.33 | 1.33% | 25.97 | 0.31 | 1.18% |
| Reference RP | 24.99 | 0.29 | 1.17% | 26.3 | 0.42 | 1.61% |
| | 25.2 | 0.22 | 0.86% | 26.42 | 0.38 | 1.42% |

### Comparison of Freshly Collected Samples and Frozen Samples

We have compared fresh vs. frozen samples side-by-side. The “fresh vs. frozen study protocol” is provided as an attachment in the current Amendment Response. There were no differences between fresh and frozen samples, and there was no bad impact of the frozen step on the samples (Table 28). Its PPA was 100% (95% CI: 0.858-1.00) and NPA was 100% (95% CI: 0.858-1.00).

TABLE 28
Comparison of Fresh versus Frozen Clinical Sample

| | Fresh Sample | | | | | |
|---|---|---|---|---|---|---|
| | CDC Monkeypox | | Frozen Sample | | | |
| | generic PCR | | QuantiVirus ™ MPXV Test | | | |
| Sample | MPXV | | ¹ J2L | ¹ B6R | ¹ RP | |
| ID | (Cq) | Call | (Cq) | (Cq) | (Cq) | Call |
| z007014 | 19.1 | Positive | 15.1 | 19.6 | 26.0 | Positive |
| z007015 | 21.2 | Positive | 18.6 | 22.8 | 21.1 | Positive |
| z007016 | 20.1 | Positive | 16.6 | 21.0 | 23.0 | Positive |
| z007017 | 17.0 | Positive | 14.0 | 18.3 | 20.2 | Positive |
| z007018 | 23.9 | Positive | 20.9 | 25.7 | 29.2 | Positive |
| z007019 | 18.7 | Positive | 15.2 | 19.5 | 23.3 | Positive |
| z007020 | 28.1 | Positive | 25.4 | 29.7 | 29.1 | Positive |
| z007021 | 23.1 | Positive | 18.7 | 23.2 | 24.8 | Positive |
| z007022 | 20.3 | Positive | 18.9 | 22.4 | 31.0 | Positive |
| z007023 | 19.3 | Positive | 15.2 | 19.3 | 19.2 | Positive |
| z007024 | 27.9 | Positive | 24.4 | 28.7 | 28.9 | Positive |
| z007025 | 33.3 | Positive | 25.6 | 30.0 | 29.9 | Positive |
| z007026 | 45.0 | Negative | 45.0 | 45.0 | 25.1 | Negative |
| z007027 | 45.0 | Negative | 45.0 | 45.0 | 20.2 | Negative |
| z007028 | 45.0 | Negative | 45.0 | 45.0 | 25.0 | Negative |
| z007029 | 45.0 | Negative | 45.0 | 45.0 | 20.2 | Negative |
| z007030 | 45.0 | Negative | 45.0 | 45.0 | 25.1 | Negative |
| z007031 | 45.0 | Negative | 45.0 | 45.0 | 30.0 | Negative |
| z007032 | 45.0 | Negative | 45.0 | 45.0 | 20.2 | Negative |
| z007033 | 45.0 | Negative | 45.0 | 45.0 | 27.7 | Negative |
| z007034 | 45.0 | Negative | 45.0 | 45.0 | 18.9 | Negative |
| z007035 | 45.0 | Negative | 45.0 | 45.0 | 18.9 | Negative |
| z007036 | 45.0 | Negative | 45.0 | 45.0 | 19.4 | Negative |
| z007037 | 45.0 | Negative | 45.0 | 45.0 | 35.3 | Negative |
| Z007338 | 45.0 | Negative | 45.0 | 45.0 | 22.2 | Negative |
| Z007339 | 23.3 | Positive | 20.5 | 23.7 | 28.7 | Positive |
| Z007340 | 17.5 | Positive | 15.3 | 19.1 | 23.1 | Positive |
| Z007341 | 21.0 | Positive | 20.0 | 23.4 | 21.6 | Positive |
| Z007342 | 45.0 | Negative | 45.0 | 45.0 | 26.5 | Negative |
| Z007343 | 19.0 | Positive | 17.3 | 21.2 | 21.0 | Positive |
| Z007344 | 45.0 | Negative | 45.0 | 45.0 | 24.7 | Negative |
| Z007345 | 45.0 | Negative | 45.0 | 45.0 | 33.0 | Negative |
| Z007346 | 45.0 | Negative | 45.0 | 45.0 | 28.8 | Negative |
| Z007347 | 21.9 | Positive | 22.1 | 26.0 | 23.5 | Positive |
| Z007348 | 20.0 | Positive | 18.8 | 22.8 | 22.0 | Positive |
| Z007349 | 17.6 | Positive | 16.4 | 20.2 | 21.3 | Positive |
| Z007350 | 45.0 | Negative | 45.0 | 45.0 | 28.5 | Negative |
| Z007351 | 45.0 | Negative | 45.0 | 45.0 | 30.9 | Negative |
| Z007352 | 26.7 | Positive | 25.6 | 29.4 | 30.6 | Positive |
| Z007353 | 45.0 | Negative | 45.0 | 45.0 | 27.0 | Negative |
| Z007354 | 25.5 | Positive | 23.7 | 27.4 | 21.4 | Positive |
| Z007355 | 19.5 | Positive | 17.3 | 21.0 | 24.4 | Positive |
| Z007356 | 45.0 | Negative | 45.0 | 45.0 | 23.0 | Negative |
| Z007357 | 45.0 | Negative | 45.0 | 45.0 | 25.5 | Negative |
| Z007358 | 20.1 | Positive | 18.4 | 22.2 | 23.8 | Positive |
| Z007359 | 45.0 | Negative | 45.0 | 45.0 | 24.5 | Negative |
| Z007360 | 23.9 | Positive | 22.5 | 26.4 | 26.5 | Positive |
| Z007361 | 45.0 | Negative | 45.0 | 45.0 | 31.9 | Negative |
| Z007362 | 32.4 | Positive | 31.3 | 35.3 | 22.4 | Positive |
| Z007363 | 45.0 | Negative | 45.0 | 45.0 | 32.1 | Negative |
| Z007364 | 17.2 | Positive | 15.1 | 18.6 | 30.8 | Positive |
| Z007365 | 27.7 | Positive | 28.1 | 32.0 | 29.2 | Positive |
| Z007366 | 45.0 | Negative | 45.0 | 45.0 | 29.2 | Negative |
| Z007367 | 32.7 | Positive | 30.4 | 33.8 | 20.9 | Positive |
| Z007368 | 45.0 | Negative | 45.0 | 45.0 | 35.0 | Negative |
| Z007369 | 45.0 | Negative | 45.0 | 45.0 | 32.1 | Negative |
| Z007370 | 25.3 | Positive | 22.0 | 25.7 | 27.1 | Positive |
| Z007371 | 45.0 | Negative | 45.0 | 45.0 | 31.2 | Negative |
| Z007372 | 45.0 | Negative | 45.0 | 45.0 | 29.2 | Negative |
| Z007373 | 17.2 | Positive | 16.3 | 20.2 | 20.1 | Positive |

¹ For QuantiVirus MPXV Test, there are 3 target genes - J2L, B6R and RP. J2L and B6R are the target monkeypox viral genes. RP is not a pathogen specific gene, but a human gene used as an internal control for the confirmation of validity of sample collection and extraction

### Clinical Performance

We have applied leftover 30 positive and 30 negative lesion samples from San Francisco Health Department and went through viral DNA extraction and QuantiVirus MPXV Test (Study DIA.0018). The study protocol and line data in EXCEL file are provided as attachments in the current EUA application. The results show the Table 29 and Table 30. Its PPA is 100% (95% CI: 0.858-1.00) and NPA is 100% (95% CI: 0.858-1.00).

TABLE 29
Monkeypox Lesion Swab Clinical Samples Testing with QuantiVirus ™ MPXV Test

| Sample | CDC Monkeypox generic PCR | | QuantiVirus ™ MPXV Test Kit | | | |
|---|---|---|---|---|---|---|
| ID | Ct | Call | J2L(FAM) | B6R(HEX) | RP(Cy5) | Call |
| z007014 | 19.08 | Positive | 15.10 | 19.55 | 25.95 | Positive |
| z007015 | 21.19 | Positive | 18.57 | 22.77 | 21.13 | Positive |
| z007016 | 20.12 | Positive | 16.56 | 21.04 | 23.04 | Positive |
| z007017 | 16.99 | Positive | 14.01 | 18.29 | 20.24 | Positive |
| z007018 | 23.94 | Positive | 20.93 | 25.69 | 29.18 | Positive |
| z007019 | 18.67 | Positive | 15.15 | 19.50 | 23.29 | Positive |
| z007020 | 28.07 | Positive | 25.38 | 29.68 | 29.07 | Positive |
| z007021 | 23.05 | Positive | 18.65 | 23.15 | 24.84 | Positive |
| z007022 | 20.27 | Positive | 18.87 | 22.44 | 31.03 | Positive |
| z007023 | 19.34 | Positive | 15.15 | 19.30 | 19.18 | Positive |
| z007024 | 27.93 | Positive | 24.42 | 28.65 | 28.88 | Positive |
| z007025 | 33.27 | Positive | 25.59 | 30.03 | 29.86 | Positive |
| z007026 | N/A | Negative | | | 25.09 | Negative |
| z007027 | N/A | Negative | | | 20.20 | Negative |
| z007028 | N/A | Negative | | | 25.03 | Negative |
| z007029 | N/A | Negative | | | 20.16 | Negative |
| z007030 | N/A | Negative | | | 25.12 | Negative |
| z007031 | N/A | Negative | | | 29.95 | Negative |
| z007032 | N/A | Negative | | | 20.23 | Negative |
| z007033 | N/A | Negative | | | 27.66 | Negative |
| z007034 | N/A | Negative | | | 18.86 | Negative |
| z007035 | N/A | Negative | | | 18.91 | Negative |
| z007036 | N/A | Negative | | | 19.43 | Negative |
| z007037 | N/A | Negative | | | 35.32 | Negative |
| Z007338 | | Negative | | | 22.16 | Negative |
| Z007339 | 23.29 | Positive | 20.54 | 23.74 | 28.71 | Positive |
| Z007340 | 17.51 | Positive | 15.25 | 19.10 | 23.13 | Positive |
| Z007341 | 21.04 | Positive | 19.96 | 23.39 | 21.58 | Positive |
| Z007342 | | Negative | | | 26.54 | Negative |
| Z007343 | 18.98 | Positive | 17.30 | 21.19 | 21.04 | Positive |
| Z007344 | | Negative | | | 24.70 | Negative |
| Z007345 | | Negative | | | 33.04 | Negative |
| Z007346 | | Negative | | | 28.83 | Negative |
| Z007347 | 21.89 | Positive | 22.10 | 25.99 | 23.52 | Positive |
| Z007348 | 20.04 | Positive | 18.76 | 22.75 | 22.04 | Positive |
| Z007349 | 17.64 | Positive | 16.38 | 20.19 | 21.32 | Positive |
| Z007350 | | Negative | | | 28.46 | Negative |
| Z007351 | | Negative | | | 30.94 | Negative |
| Z007352 | 26.67 | Positive | 25.62 | 29.42 | 30.61 | Positive |
| Z007353 | | Negative | | | 26.96 | Negative |
| Z007354 | 25.53 | Positive | 23.66 | 27.38 | 21.41 | Positive |
| Z007355 | 19.49 | Positive | 17.30 | 21.49 | 24.36 | Positive |
| Z007356 | | Negative | | | 23.03 | Negative |
| Z007357 | | Negative | | | 25.50 | Negative |
| Z007358 | 20.11 | Positive | 18.36 | 22.18 | 23.79 | Positive |
| Z007359 | | Negative | | | 24.45 | Negative |
| Z007360 | 23.86 | Positive | 22.54 | 26.41 | 26.53 | Positive |
| Z007361 | | Negative | | | 31.88 | Negative |
| Z007362 | 32.4 | Positive | 31.29 | 35.25 | 22.39 | Positive |
| Z007363 | | Negative | | | 32.14 | Negative |
| Z007364 | 17.21 | Positive | 15.12 | 18.63 | 30.75 | Positive |
| Z007365 | 27.71 | Positive | 28.07 | 32.00 | 29.20 | Positive |
| Z007366 | | Negative | | | 29.23 | Negative |
| Z007367 | 32.74 | Positive | 30.39 | 33.82 | 20.92 | Positive |
| Z007368 | | Negative | | | 35.00 | Negative |
| Z007369 | | Negative | | | 32.06 | Negative |
| Z007370 | 25.25 | Positive | 22.02 | 25.65 | 27.13 | Positive |
| Z007371 | | Negative | | | 31.20 | Negative |
| Z007372 | | Negative | | | 29.18 | Negative |
| Z007373 | 17.17 | Positive | 16.31 | 20.15 | 20.09 | Positive |

TABLE 30
Summary of Clinical Test of QuantiVirus MPXV Test

| | CDC Monkeypox | | | |
|---|---|---|---|---|
| QuantiVirus ™ | generic PCR | | | |
| MPXV Test | Positive | Negative | PPA (%) | NPA (%) |
| Positive | 30 | 0 | 100% | 100% |
| Negative | 0 | 30 | (95% CI: | (95% CI: |
| Total | 30 | 30 | 0.858-1.00) | 0.858-1.00) |

All patents, patent applications and publications cited in this application including all cited references in those patents, applications and publications, are hereby incorporated by reference in their entirety for all purposes to the same extent as if each individual patent, patent application or publication were so individually denoted.

Although the foregoing description (Angres) contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

What is claimed is:

1. A PCR primer set useful for detecting Monkeypox virus selected from the group consisting of the following primer sets: (a) a primer set comprising a primer consisting of MPXVF SEQ ID NO: 1 GGAAAATGTAAAGACAACGAATACAG and a primer MPXVR SEQ ID NO: 2 GCTATCACATAATCTGGAAGCGTA; (b) a primer set comprising a primer consisting of B6RF SEQ ID NO: 4 AATGGCGTTGACAATTATGGGTG and a primer consisting of B6RR SEQ ID NO: 5 ATTGGTCATTATTTTTGTCACAGGAACA; and (c) a primer set comprising a primer consisting of RNasePF SEQ ID NO: 7 AGATTTGGACCTGCG AGCG and a primer consisting of RNasePR SEQ ID NO: 8 GAGCGGCTGTCTCCACAAGT; wherein the primer set specifically amplifies a target region of Monkeypox virus in a polymerase chain reaction (PCR).

2. Oligonucleotides, for use as a probe to detect the amplified nucleic acid sequence resulting in the amplification of a target sequence located within the genome of Monkeypox virus, said amplification being based on pair of oligonucleotides according to claim 1, said probe being selected from the group consisting of MPXVPr (Probe) SEQ ID NO: 3 AAGCCGTAATC TATGTT; B6RPr (Probe); SEQ ID NO: 6 AGAGATTAGAAATA and RNasePPr (Probe) SEQ ID NO: 9 TTCTGACCTGAAGGCTCTGCGCG.

3. A method for determining the presence or absence of Monkeypox virus in a biological sample, the method comprising: (a) contacting nucleic acid from a biological sample with at least one primer which is a nucleic acid of claim 1, (b) subjecting the nucleic acid and the primer to amplification conditions, and (c) determining the presence or absence of amplification product, wherein the presence of amplification product indicates the presence of RNA associated with Monkeypox virus in the sample.

4. A method for detecting Monkeypox virus by contacting a biological sample with a set of primers and a probe, incubating under conditions allowing amplification of nucleic acid using said primers, and determining binding of said probe to amplified nucleic acid, wherein detecting binding of said probe to amplified nucleic acid indicates the presence of Monkeypox associated virus, wherein the the primers are selected from the group consisting of the following primer sets: (a) a primer set comprising a primer consisting of MPXVF SEQ ID NO: 1 GGAAAATGT AAAGACAACGAATACAG and a primer MPXVR SEQ ID NO: 2 GCTATCACATAATCT GGAAGCGTA; (b) a primer set comprising a primer consisting of B6RF SEQ ID NO: 4 AATGGCGTTGACAATTATGGGTG and a primer consisting of B6RR SEQ ID NO: 5 ATTGGTCATTATTTTTGTCACAGGAACA; and (c) a primer set comprising a primer consisting of RNasePF SEQ ID NO: 7 AGATTTGGACCTGCG AGCG and a primer consisting of RNasePR SEQ ID NO: 8 GAGCGGCTGTCTCCACAAGT; and wherein the probe is selected from the group consisting of MPXVPr (Probe) SEQ ID NO: 3 AAGCCGTAATC TATGTT; B6RPr (Probe); SEQ ID NO: 6 AGAGATTAGAAATA and RNasePPr (Probe) SEQ ID NO: 9 TTCTGACCTGAAGGCTCTGCGCG; and wherein the probe is labeled with two dyes, one dye of which is a fluorescent reporter dye, and one dye of which is a quencher dye, and wherein at least one dye is a fluorescent dye; and the Monkeypox virus is detected by detection of real time fluorescence, if amplification of virus specific sequence occurs.

5. The method of claim 4, wherein the amplification and detection are performed using real time RT-PCR.

6. The method according to claim 4, wherein the primer set are MPXVF SEQ ID NO: 1 GGA AAATGTAAAGACAACGAATACAG and a primer MPXVR SEQ ID NO: 2 GCTATCACAT AATCTGGAAGCGTA and wherein the probe has the sequence shown as MPXVPr (Probe) SEQ ID NO: 3 AAGCCGTAATCTATGTT.

7. The method according to claim 4, wherein the reporter dye is FAM, 6-FAM, 5-FAM and ALEXA-288.

8. The method according to claim 4, wherein the quencher dye is TAMRA, DABCYL or QSY.

9. The method according to claim 4, wherein detection is quantitative detection of the real time fluorescence signal intensity.

10. The method according to claim 4, wherein the biological sample is from a skin lesion.

11. The method according to claim 10, wherein the biological sample is selected from the group consisting of lesion exudate, lesion roofs or lesion crusts.

12. A kit for detecting Monkeypox virus in a biological sample comprising a PCR primer set selected from the group consisting of the following primer sets: (a) a primer set comprising a primer consisting of MPXVF SEQ ID NO: 1 GGAAAATGT AAAGACAACGAATACAG and a primer MPXVR SEQ ID NO: 2 GCTATCACATAATCT GGAAGCGTA; (b) a primer set comprising a primer consisting of B6RF SEQ ID NO: 4 AATGGCGTTGACAATTATGGGTG and a primer consisting of B6RR SEQ ID NO: 5 ATTGGTCATTATTTTTGTCACAGGAACA; and (c) a primer set comprising a primer consisting of RNasePF SEQ ID NO: 7 AGATTTGGACCTGCG AGCG and a primer consisting of RNasePR SEQ ID NO: 8 GAGCGGCTGTCTCCACAAGT; wherein the primer set specifically amplifies a target region of Monkeypox virus in a polymerase chain reaction (PCR).

13. The kit of claim 12, further including a probe selected from the group consisting of MPXVPr (Probe) SEQ ID NO: 3 AAGCCGTAATC TATGTT; B6RPr (Probe); SEQ ID NO: 6 AGAGATTAGAAATA and RNasePPr (Probe) SEQ ID NO: 9 TTCTGACCTGAAGGCTCTG CGCG.

14. The kit of claim 13, further including a reporter dye selected from the group consisting of FAM, 6-FAM, 5-FAM and ALEXA-288.

15. The kit of claim 13, further including a quencher dye selected from the group consisting of TAMRA, DABCYL or QSY.

16. The Kit according to claim 12, further comprising enzymes and reagents required for performing a real time RT-PCR reaction.

17. The method of claim 4, wherein the primers/probes are specific to the J2L and B6R Monkeypox genomic region.

18. The kit of claim 13, wherein the primers/probes are specific to the J2L and B6R Monkeypox genomic region.

19. The method of claim 4, wherein said method includes a Positive Control (PC), an Extraction Control (EC) and a No Template Control (NTC).

20. The kit of claim 13, wherein said kit further includes a Positive Control (PC), an Extraction Control (EC) and a No Template Control (NTC).
